(19) United States
(12) Patent Application Publication
(10) Pub. No.: US 2009/0265116 A1
(43) Pub. Date: Oct. 22, 2009

(54) PREDICTION OF AN INDIVIDUAL'S RISK OF DEVELOPING RHEUMATOID ARTHRITIS

(75) Inventor: Michael J. Walsh, San Diego, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: Cypress Bioscience, Inc., San Diego, CA (US)

(21) Appl. No.: 12/428,299

(22) Filed: Apr. 22, 2009

Related U.S. Application Data
(60) Provisional application No. 61/047,094, filed on Apr. 22, 2008.

(51) Int. Cl. G06F 19/00; G01N 33/48

(52) U.S. Cl. 702/19

(57) ABSTRACT

Methods for predicting the likelihood of development of rheumatoid arthritis for individuals that present with recent-onset undifferentiated arthritis. The methods are based on the determination of a set of clinical markers and/or parameters and determining a predicted risk for developing rheumatoid arthritis. Clinical markers and parameters that are decisive for the risk for developing rheumatoid arthritis may include serum levels of C-reactive protein, Rheumatoid factors, anti-CCP antibodies, anti-MCV as well as age, gender, localization of the joint complaints, length of morning stiffness, and number of tender and/or swollen joints or combinations thereof. The method may be performed by a computer. The invention further relates to a computer, a sample analyser and a computer program product for performing the method and a data carrier with the computer program product.

### CROSS-REFERENCE TO RELATED APPLICATIONS

This application claims the benefit of U.S. Provisional Application Ser. No. 61/047,094, filed on Apr. 22, 2008, entitled “PREDICTION OF AN INDIVIDUAL'S RISK OF DEVELOPING RHEUMATOID ARTHRITIS,” the disclosure of which is incorporated herein by reference in its entirety.

### FIELD OF THE INVENTION

The present invention relates to predicting the likelihood of developing rheumatoid arthritis in individuals with undiagnosed or undifferentiated arthritis. In particular, the present invention relates to using various clinical parameters to differentially diagnose or predict the development of rheumatoid arthritis.

### BACKGROUND OF THE INVENTION

Rheumatoid arthritis (RA) is a chronic disease of the joints and is characterized by inflammation of the synovium which can subsequently result in erosive destruction of the joints. RA affects over 1.3 million Americans. Prevalence of RA worldwide was estimated to be over 20 million in 2004. The cause of RA is presently unknown though many theories have been proposed. Indefinite and continuous RA can result in a systemic problem that affects other organs of the individual with RA. Because of the chronic, painful, and debilitating nature of the disease which can progress to a systemic disease, early diagnosis and treatment is therefore of critical importance. However, the early diagnosis of RA present a major issue for caregivers such as the physicians because the early symptoms of RA are very similar to other forms of arthritis. Furthermore, many individuals remain undiagnosed until onset of the disease where much of the joints have been destroyed or eroded because these individuals do not manifest clinical characteristics that are classifiable as symptomatic of RA.

Many individuals in outpatient clinic with recent-onset arthritis are referred to as having early arthritis. Some of these individuals may, at first presentation, have a disease that can be classified according to current arthritis evaluation criteria. For example, individuals may be directly diagnosed with rheumatoid arthritis or reactive arthritis. Reactive arthritis is an acute form of arthritis which occurs after a viral or bacterial infection that spontaneously disappears in several weeks or months, and which features the following three conditions: (1) inflamed joints; (2) inflammation of the eyes (conjunctivitis); and (3) inflammation of the genital, urinary or gastrointestinal system. On the other hand, other individuals may present with an early arthritis that cannot be directly classified. These patients are considered to have an undifferentiated arthritis (UA), which is defined as an early arthritis for which, according to the available classification criteria, no diagnosis can be made.

When individuals at first presentation are diagnosed with RA or reactive arthritis, prediction of whether the disease will become persistent or erosive is straightforward, as most RA patients will have a persistent and erosive disease course, while most patients with reactive arthritis will have a self-limiting disease course which in most cases, does not recur.

In contrast, while some individual with UA remit spontaneously, about one third will develop RA. Treatment with methotrexate in individuals with UA is known to inhibit progression to RA and inhibit joint damage. However, because of the potential toxicity associated with methotrexate and other DMARDs, only individuals who have a high risk of developing RA, not those who are likely to remit spontaneously, should be treated with these agents. Thus, a method for predicting which patients with UA are most likely to develop RA would be exceedingly beneficial since only those most likely to develop RA would be exposed to potentially toxic therapeutic agents.

Although Morel and Combe (2005, Best Practice & Research Clinical Rheumatology 19:137-146) reviewed factors associated with the development of RA, or associated with the development of erosions in patients already diagnosed with the disease, the reference does not disclose a predictive model capable of assessing whether a patient with UA will develop RA.

In addition, several prognostic models that allow prediction of arthritis outcome have been described (e.g. Visser et al., 2002, Arthritis Rheum. 46:357-365; Visser, 2005, Best Practice & Research Clinical Rheumatology 19:55-72). However, the cohorts used to build and validate the models were made up of individuals with early arthritis, including those classified with RA and reactive arthritis diagnoses, as well as those with UA. Furthermore these studies were used to build model with the objective of determining disease progression (erosive disease in particular), rather than differentiating RA from UA. Thus, these models are not capable of assisting in the differential diagnosis of patients that present with UA, and cannot be used to predict development of RA in patients with UA. Accordingly, there is a need for a method predicting whether patients with UA will develop RA in order to provide the individual with individualized therapy before the disease progresses to the chronic and debilitating form of arthritis.

### SUMMARY OF THE INVENTION

The present invention is based, in part, on the discovery that certain clinical parameters and/or markers are useful for predicting the likelihood of developing RA in individuals with UA. Accordingly, the present invention provides methods, systems, combination of tests, and collection of results useful for predicting whether an individual with UA will develop RA.

In one aspect, the present invention relates to a method of predicting whether an individual with undifferentiated arthritis will develop rheumatoid arthritis by determining the presence or absence of antibodies to mutated citrullinate vimentin (anti-MCV antibody) in the individual, where the presence of anti-MCV antibody in the individual with undifferentiated arthritis is indicative of the risk of the individual for developing rheumatoid arthritis.

In one embodiment, the method further includes determining physical symptoms such as, but not limited to the duration of morning stiffness of the individual. The duration of morning stiffness correlates with the risk of the individual for developing rheumatoid arthritis.

In another embodiment, the method further includes determining at least one clinical parameter of the individual, e.g. 1) age, 2) gender, 3) distribution of involved joints, 4) duration of morning stiffness, 5) number of tender joints, and 6) number of swollen joints. In some embodiments, a risk value for one or more clinical parameters can be assigned based on a predefined risk value index for the clinical parameter for predicting the risk of the individual of developing rheumatoid arthritis, e.g., in combination with a risk value assigned for the presence or absence of anti-MCV antibody in the individual.

In a further embodiment, the method includes determining the presence or absence of at least one additional clinical marker, e.g., the presence or absence of antibodies to certain clinical markers, other than antibodies to MCV. Examples of antibodies include antibodies to cyclic-citrullinated peptide (anti-CCP antibody), antibodies to Rheumatoid Factor (RF autoantibody), where the presence of anti-CCP antibody or RF autoantibody correlates with the risk of the individual for developing rheumatoid arthritis.

In another further embodiment, the method includes determining the serum level of certain clinical markers. Examples of clinical markers include C-reactive protein (CRP), high sensitivity C-reactive protein (HS CRP) and erythrocyte sedimentation rate (ESR). In some embodiments, the level of clinical markers can be assigned a risk value based on a predefined risk value index for the clinical marker and such risk value can be used in combination with the risk value assigned to the presence or absence of anti-MCV to predict the risk of the individual developing rheumatoid arthritis.

In another aspect, the invention provides a computer having a processor and a memory, where the processor is arranged to read from the memory and write into the memory. In one embodiment, the memory comprises data obtained using the various clinical parameters and/or markers, and instructions arranged in such manner as to provide the processor with the capacity to perform the method of predicting whether an individual with undifferentiated arthritis will develop rheumatoid arthritis.

In yet another aspect, the invention provides a system for determining a predicted risk of an individual with undifferentiated arthritis in developing rheumatoid arthritis. In one embodiment, the system comprises means for receiving a characteristic of a clinical parameter such as but not limited to the 1) age, 2) gender, 3) distribution of involved joints, 4) duration of morning stiffness, 5) number of tender joints, and 6) number of swollen joints. The system also comprises means for receiving a characteristic of a first clinical marker comprising anti-MCV antibody and optionally a second clinical marker selected from the group consisting of anti-CCP antibody, Rheumatoid factor autoantibody (IgA, IgM, and/or IgG), C-reactive protein (CRP), high sensitivity C-reactive protein (HS CRP) and erythrocyte sedimentation rate (ESR). In some embodiments, the system further includes means for assigning a risk value to each characteristic of clinical parameters and clinical markers; and means for determining a predicted risk of the individual developing rheumatoid arthritis based at least partly on the assigned risk values.

In a further aspect, the invention provides a system for determining a predicted risk of an individual with undifferentiated arthritis developing rheumatoid arthritis, the system includes a blood sample analyzer configured to analyze a blood sample of the individual for the presence or absence of a first clinical marker of anti-MCV antibody, and optionally a second clinical marker selected from the group consisting of anti-CCP antibody, RF autoantibody, C-reactive protein (CRP), high sensitivity C-reactive protein (HS CRP) and erythrocyte sedimentation rate (ESR); and a computing device configured to assign a risk value to each of the clinical marker determined by the blood sample analyzer based on predefined risk values associated with each clinical marker stored in a memory, and to determine a predicted risk of the individual developing rheumatoid arthritis based at least partly on the collection of the risk value assigned to each of the clinical marker.

In another aspect of the invention, a combination of tests useful for predicting whether an individual with undifferentiated arthritis will develop rheumatoid arthritis is provided. The combination tests include a first test for the presence or absence of anti-MCV antibodies and a second test. The combination tests can include a plurality of tests. In one embodiment, the combination tests include a first test and a second test. In another embodiment, the combination tests include a first, a second test and a third test. In a further embodiment, the combination tests include a first test, a second test, a third test and a fourth test. The second, third and/or fourth tests include but not limited to tests for the serum level of C-reactive protein, HS-CRP or ESR, tests for the presence or absence of RF autoantibody, and tests for the presence or absence of anti-CCP antibodies.

In one embodiment the test for the presence or absence of anti-MCV antibodies include using a peptide derived from native vimentin where the peptide comprises at least one additional amino acid residue, e.g., an arginine or modified arginine compared to the native sequence. In a further embodiment, the tests for the presence or absence of anti-MCV antibodies include using a peptide derived from native vimentin where the peptide comprises at least one additional arginine residue in at least one of positions 16, 17, 19, 41, 58, 59, 60, 68, 76, 140, 142, 147, 363, 406 or 452.

In another aspect, the invention relates to a method of providing useful information for predicting whether an individual with undifferentiated arthritis will develop rheumatoid arthritis. The method comprises determining a set of clinical markers for the individual and providing to an entity that combines the set of clinical markers with a set of clinical parameters for predicting development of rheumatoid arthritis. The set of clinical markers include the presence or absence of anti-MCV antibodies and at least one other clinical marker value such as but not limited to the serum level of C-reactive protein, HS-CRP or ESR, the presence or absence of RF autoantibody, and/or the presence or absence of anti-CCP antibodies.

In one embodiment the test for the presence or absence of anti-MCV antibodies include using a peptide derived from native vimentin and/or variants thereof, where the peptide comprises at least one additional amino acid residue compared to the native sequence. The additional amino acid residue can be an arginine or modified arginine. In a further embodiment, the tests for the presence or absence of anti-MCV antibodies include using a peptide derived from native vimentin and/or variants thereof, where the peptide comprises at least one additional arginine residue in at least one of positions 16, 17, 19, 41, 58, 59, 60, 68, 76, 140, 142, 147, 363, 406 or 452. In one embodiment, the set of clinical parameters include at least two or more physical characteristics or symptoms, such as but not limited to, the duration of morning stiffness, the localization of the joint complaints, the number of tender joints, the number of swollen joints, the age and the gender of the individual. In one embodiment, the entity is a clinician or a service provider.

In a further aspect, the invention provides a collection of results useful for predicting whether an individual with undifferentiated arthritis will develop rheumatoid arthritis. The collection of results includes values for a first set of clinical markers for an individual, wherein the first set of clinical markers include the presence or absence of anti-MCV antibodies and at least one clinical marker value such as but not limited to the serum level of C-reactive protein, HS-CRP or ESR, the presence or absence of Rheumatoid factor autoantibody, and the presence or absence of anti-CCP antibodies. In one embodiment, the collection of results include the presence or absence of anti-MCV antibodies detected using a peptide derived from native vimentin and/or variants thereof, where the peptide include at least one additional amino acid residue, e.g., modified or unmodified arginine residue at, for example, positions 16, 17, 19, 41, 58, 59, 60, 68, 76, 140, 142, 147, 363, 406 or 452, compared to the native sequence, In a further embodiment, the collection of results include instruction for using the first set of clinical markers in combination with a set of clinical parameters for an individual. The clinical parameters can include at least two or more physical characteristics or symptoms, such as but not limited to, the duration of morning stiffness, the localization of the joint complaints, the number of tender joints, the number of swollen joints, the age and the gender of the individual.

### BRIEF DESCRIPTION OF THE DRAWINGS

FIG. 1 shows a schematic example of an embodiment of a computer as may be used in one or more of the embodiments described.

FIG. 2 schematically depicts a flow diagram of a procedure as may be executed by the computer of FIG. 1 according to an embodiment of the invention.

FIG. 3 illustrates an exemplary table storing exemplary risk values that are associated with ranges of parameter values for several clinical parameters.

FIG. 4 illustrates an exemplary form that may be used in order to calculate risk values associated with particular parameter values.

FIG. 5 is a graph illustrating a predicted risk of developing rheumatoid arthritis as a function of the total risk value.

FIG. 6 illustrates an exemplary table storing exemplary total risk values associated with predicted risk scores.

FIG. 7 shows the predicted risk curve obtained for the re-derived prediction rule model superimposed on the predicted risk curve obtained for the original prediction rule model.

FIG. 8 shows the receiver-operator characteristic (ROC) curve for the “Enhanced” prediction rule model compared to the ROC curve for the original prediction rule model.

### DETAILED DESCRIPTION OF THE PREFERRED EMBODIMENTS

The present invention is based, in part, on the discovery that certain clinical parameters and/or markers are useful for predicting the likelihood of developing rheumatoid arthritis (RA) in individuals with undifferentiated arthritis (UA). Accordingly, the present invention provides methods, systems, combinations of tests, and collections of results useful for predicting whether an individual with UA will develop RA.

In general, UA is defined as arthritis for which no differential diagnosis can be made using available classification criteria, such as the American College of Rheumatology (ACR) 1987 classification criteria for rheumatoid arthritis. (See, e.g., Arnette et al., 1988, Arthritis Rheum. 31: 315-324). RA, on the other hand is a common severe, chronic inflammatory joint disease that can result in chronic pain, loss of function and disability in the individual afflicted with the disease.

As used herein, “antibodies” are proteins comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. Antibodies exist as intact immunoglobulins or as a number of well-characterized fragments produced by digestion with various peptidases. Antibodies to the various clinical markers of the present invention can be directed to any suitable epitope, e.g., amino acid sequences in the polypeptides or proteins or the carbohydrate moiety attached to the protein such as but not limited to sialic acid, mannoses, glucose, galactose etc.

According to one aspect, the invention provides methods of predicting whether an individual with UA will develop RA by determining the presence or absence of antibodies to vimentin, e.g., native vimentin or a variant or isoform thereof in an individual with UA where the presence or absence of the antibodies in the individual is indicative of the risk of the individual for developing RA, e.g., a risk value with respect to development of RA can be provided based on the presence or absence of the antibodies.

According to the present invention, variants or isoforms of vimentin can be full length or partial fragments of vimentin, e.g., fragments of vimentin that are immunologically reactive. In one embodiment, variants or isoforms of vimentin are mutated vimentin having one or more amino acid additions, deletions and/or substitutions in a native or wild type vimentin. In some embodiments, variants or isoforms of vimentin are vimentins with one or more modified amino acids, e.g., citrullinated amino acids. In some embodiments, variants or isoforms of vimentin include vimentins with citrullinated amino acids and one additional mutation. In some other embodiments, variants or isoforms of vimentin are vimentins with one or more citrullines. Citrulline is arginine that has been post-translationally modified (de-iminated) by a family of enzymes called peptidylarginine deaminase (PAD). In some other embodiments, variants or isoforms of vimentin are vimentins with one or more post translational modifications. In general, post translational modifications include citrullination, methylation, glycosylation, lipoylation, amidation, sulfation, acetylation, glutamylation, selenation, biotinylation, isoprenylation, alkylation, etc.

In general, mutated citrullinated vimentin (MCV) includes vimentin that contains at least one citrullinated amino acid residue and a mutation, either at a separate position or co-localized with the citrullinated amino acid. In one embodiment, MCV includes vimentin that contains at least one citrulline, e.g., citrullinated arginine and a mutation, e.g., insertion(s) of one or more amino acids including without limitation arginine, leucine, proline, threonine, tyrosine, etc. In another embodiment, MCV includes vimentin that contains at least one citrulline and a mutation of one or more arginine insertions to the wild type vimentin, with or without modification such as citrullination. In another embodiment, MCV includes vimentin that contains at least one citrulline and a mutation of one or more arginine insertions via substituting one or more amino acids in the wild type vimentin. In yet another embodiment, MCV includes vimentin that contains at least one citrulline and where the citrulline is within a mutation of the wild type vimentin, e.g., a vimentin with an arginine inserted into the wild type vimentin either via simple insertion or insertion and substituting out of an existing amino acid in the wild type vimentin and where the inserted arginine is citrullinated.

In some embodiments, MCV includes vimentin comprising at least one additional unmodified arginine residue or a citrulline. The additional unmodified arginine residue or citrulline can be at positions, such as but not limited to, positions 16, 17, 19, 41, 58, 59, 60, 68, 76, 140, 142, 147, 363, 406 or 452 of the native vimentin protein sequence. In one embodiment, at least one arginine in the form of citrulline, can be, for example, in at least one of positions 4, 12, 23, 28, 36, 45, 50, 64, 71, 100, 320, 364 or 378. In one embodiment, the preferred positions are 41, 58, 59 and/or 60.

In some other embodiments, MCV includes vimentin having one or more insertions of amino acids including arginine, leucine, proline, threonine, tyrosine, lysine, histidine, alanine, cysteine, aspartic acid, glutamic acid, phenylalanine, glycine, isoleucine, methionine, asparagine, glutamine, serine, valine, trytophan or a combination thereof. In some other embodiments, MCV includes vimentin having an additional leucine residue inserted in at least one of positions 3, 20, 33, 36, 37, 94, 165, 361, 399 or 426, preferably in positions 33, 36 and/or 37 of the native vimentin with or without any arginine insertion. In some other embodiments, MCV includes vimentin having an additional proline residue inserted in at least one of positions 21, 41, 43, 50, 54, 62, 64 or 89, preferably in positions 41, 43, 50, 54, 62 and/or 64 of the native vimentin, with or without any arginine insertion. In yet some other embodiments, MCV includes vimentin having an additional threonine residue inserted in at least one of positions 24, 35 or 99 of the native vimentin, with or without any arginine insertion. In some further embodiments, MCV includes vimentin having an additional tyrosine residue inserted in at least one of positions 25, 39, 42, 49, 55 or 67 of the native vimentin, with or without arginine insertion.

In the context of the present disclosure, determining the presence or absence of anti-MCV antibodies can be either quantitatively (e.g., low or high levels, etc.) or qualitatively, using any suitable methods known or later discovered, e.g., point of care rapid tests or tests conducted in labs. For example, one can use the anti-MCV assay commercially available from Orgentec Diagnostika GMBH (Mainz, Germany), e.g., Rheumachec®, a rapid lateral flow immunochromatographic assay or methods based on ELISA. Briefly, MCV can be immobilized on a solid surface and provided in a condition for binding to MCV antibodies in a sample of an individual. Such binding can be detected by any suitable means, e.g., conjugated secondary antibody such as horse-radish peroxidase (HRP) conjugated anti-human IgG, etc.

MCV and assays for detecting MCV is also described in WO2007/000320, which is incorporated herein by reference in its entirety.

According to the present invention, the presence of anti-MCV antibody in an individual with UA is indicative of the risk of the individual for developing RA. Such indication can be represented by any suitable means and provided in any suitable form. For example, such risk indication can be represented qualitatively as high (higher than normal) level of risk or quantitatively such as by assigning a risk value based on a predetermined risk index value, e.g., values established based on the degree of correlation between the presence of anti-MCV antibodies and development of RA. In one embodiment, a risk value is assigned to the presence or absence of anti-MCV antibody when such clinical marker is considered in combination with other related clinical markers or parameters.

According to another embodiment of the present invention, in addition to detecting the presence or absence of anti-MCV antibodies, one or more additional clinical markers can be used in combination with the clinical marker of anti-MCV for predicting the risk of an individual for developing RA. In one embodiment, the additional clinical markers include any clinical marker related to RA, e.g., marker(s) for RA diagnostics, monitoring of RA progression, monitoring of RA treatment, and/or RA prognosis. In another embodiment, the additional clinical markers include without any limitation anti-CCP antibody, Rheumatoid Factor (RF) autoantibody, anti-nuclear antibody, antibodies against any citrullinated proteins or polypeptides (other than anti-MCV), level of C-reactive protein (CRP), high sensitivity C-reactive protein (HS CRP), and erythrocyte sedimentation rate (ESR).

In yet another embodiment, the additional clinical marker includes antibodies against any citrullinated proteins or polypeptides, e.g., antibodies against a protein or polypeptide with one or more citrullines. In yet another embodiment, the additional clinical marker includes antibodies against any citrullinated proteins or polypeptides, e.g., antibodies against cyclic citrullinated proteins (CCP) such as but not limited to CCP1, CCP2 and CCP3, myelin basic protein, filaggrin, histone, fibrin, keratin and/or variants thereof.

In general, any detection of anti-CCP antibodies is indicative of the presence of anti-CCP antibodies. In one exemplary embodiment, antibodies to CCP are considered to be present in a sample from an individual if there is at least 10, 20 or 25 units of antibody as measured using the ELISA. (Immunoscan RA Mark 2, obtainable from Euro-Diagnostica, Arnhem, The Netherlands). Other exemplary suitable tests for anti-CCP are described by van Venrooij and van de Putte (2003, Ned Tijdschr Geneeskd. 147(5):191-4).

According to the present invention, the anti-CCP antibodies (anti-CCP1, anti-CCP2, anti-CCP 3) may be of any isotype, including IgG (e.g., IgG1, IgG2, IgG3 and IgG4), IgA and IgM. In one embodiment, the anti-CCP antibody is of IgM, IgG2, and/or IgG3 isotype. In another embodiment, determining the presence or absence of anti-CCP includes determining the isotype pattern of anti-CCP. For example, in general a diverse pattern (versus a less diverse pattern or any isotype pattern that is biased towards certain anti-CCP isotype(s) such as IgM, IgG2, and/or IgG3) can be indicative of risk for developing RA.

According to the present invention, the presence of anti-CCP antibody in an individual with UA is indicative of the risk of the individual for developing RA. Such indication can be represented by any suitable means and provided in any suitable form. For example, such risk indication can be represented qualitatively as high (higher than normal) level of risk or quantitatively such as by assigning a risk value based on a predetermined risk index value, e.g., values established based on the degree of correlation between the presence of anti-CCP antibodies and development of RA. In one embodiment, a risk value is assigned to the presence or absence of anti-CCP antibody when such clinical marker is considered in combination with other related clinical markers or parameters.

In yet another embodiment, the additional clinical marker includes Rheumatoid Factor (RF) autoantibodies. Rheumatoid Factor (RF) autoantibody is an autoantibody directed against the Fc portion of the IgG antibodies. Without being limited to any particular technical aspect, the immune complexes formed between RF and IgG are considered to contribute to the progression of inflammatory diseases such as RA and/or other autoimmune diseases, for example, Sjögren's syndrome, by triggering various types of inflammation-related pathways in the body. Rheumatoid Factor (RF) autoantibodies are usually antibodies of the IgM class, although other isotypes may also be determined (e.g. IgG, IgA) in any of the methods described herein.

In general, RF autoantibody can be detected by any suitable means known or later developed. According to the present invention, any detection of RF autoantibody can be indicative of the presence of RF autoantibody in a sample. In one exemplary embodiment, RF autoantibody is considered to be present in a sample from an individual upon demonstration of abnormal amount of serum RF autoantibody, with thresholds set such that the assay is positive in less than 5% of normal subjects.

According to the present invention, the presence of RF autoantibody in an individual with UA is indicative of the risk of the individual for developing RA. Such indication can be represented by any suitable means and provided in any suitable form. For example, such risk indication can be represented qualitatively as high (higher than normal) level of risk or quantitatively such as by assigning a risk value based on a predetermined risk index value, e.g., values established based on the degree of correlation between the presence of RF autoantibody and development of RA. In one embodiment, a risk value is assigned to the presence or absence of RF autoantibody when such clinical marker is considered in combination with other related clinical markers or parameters.

In yet another embodiment, the additional clinical marker includes C-reactive protein (CRP). CRP is a prototypic acute phase protein produced in the liver and can be found in the blood in response to tissue injury and inflammation. The concentration of CRP normally can increase several-fold in response to different types of tissue damage and inflammation and is usually considered a significant disease indicator. High-sensitivity (HS)CRP is generally used to detect the risk for cardiovascular disease, but the dynamic range of concentrations measured using HS CRP can also be found in patients with UA.

In yet another embodiment, the additional clinical marker includes erythrocyte sedimentation rate (ESR), which is the rate at which red blood cells precipitate within a specified time, normally within 1 hour. Normally ESR is increased by any increase in inflammation and thus is used as an indicator of inflammation. In some embodiments, ESR is used, either instead of, or combined with the determination of CRP levels.

According to the present invention, the level of CRP and/or ESR in an individual with UA is indicative of the risk of the individual for developing RA. Such indication can be represented by any suitable means, e.g., represented quantitatively such as by assigning a risk value based on a predetermined risk value index, especially when the level of CRP and/or ESR is considered in combination with other related clinical markers or parameters. In general, higher than normal level of CRP and/or ESR is indicative of risk for developing RA.

According to the present invention, determining the presence or absence of anti-MCV or any additional clinical marker can be either quantitatively or qualitatively. For example, one can use any suitable assays known or later developed to determining the “absolute” presence or absence of the relevant clinical marker or determining the level of the relevant clinical marker wherein a level less than certain pre-determined “cut off” or “standard” level is determined as “absence” of the clinical marker. In general, detection of the presence or absence of an antibody can include either detecting based on certain detectable signal or detecting based on antibody titer. In one embodiment, detection of the presence or absence of an antibody is carried by point of care rapid tests, e.g., lateral flow immunochromatographic assays. In another embodiment, detection of the presence or absence of an antibody is carried by tests conducted in labs, e.g. ELISA. In yet another embodiment, detection of the presence or absence of an antibody includes detection of antibodies directed against sugar moieties attached to the relevant protein or polypeptide. The sugar moieties can be sialic acid, glucose, galactose and mannose.

Any suitable methods or assays can be used to detect the presence or absence of anti-MCV and additional clinical markers or detect the level of additional clinical markers. In general, antibodies can be detected via any suitable methods known or later developed, e.g., enzyme-linked immunosorbent assay (ELISA), lateral flow immunochromatographic assay, immunoturbidimetry, rapid immunodiffusion, Western blot, radioimmunoassay, chemoluminescence immunoassay and visual agglutination, etc. Detection of a protein level can be carried out either directly by measuring the protein level or indirectly by measuring the post translationally modified protein level, protein activity, mRNA level, and/or mRNA activity, etc.

According to another embodiment of the present invention, in addition to detecting the presence or absence of anti-MCV antibodies, one or more clinical parameters can be used in combination with the clinical marker of anti-MCV for predicting the risk of an individual for developing RA. In one embodiment, the clinical parameter includes any physical or clinical symptom associated with RA, e.g., symptoms associated with RA diagnostics, monitoring of RA progression, monitoring of RA treatment, and/or RA prognosis.

In another embodiment, the clinical parameter includes the duration of morning stiffness of an individual. Usually morning stiffness as a result of joint stiffness is characterized by loss of motion or loss of range of motion. Morning stiffness can also be characterized by pain on moving a joint or the severity of the pain experience by the individual. According to the present invention, the duration of morning stiffness correlates with the risk of the individual for developing rheumatoid arthritis.

In another embodiment, the clinical parameter includes the severity of morning stiffness, e.g., severity determined by measuring the pain intensity using visual analogue scale (VAS).

In yet another embodiment, the clinical parameter includes age, gender and combinations thereof. In still another embodiment, the clinical parameter includes distribution of involved joints, number of tender joints, and number of swollen joints. In still yet another embodiment, the clinical parameter includes (1) age, (2) gender, (3) distribution of involved joints, (4) duration of morning stiffness, (5) number of tender joints (6) number of swollen joints, and combinations thereof.

In some embodiments, the clinical parameter for distribution of involved joints includes the involvement of small joints in the hands and feet, the involvement is symmetrical or assymetrical, the involvement affects the upper extremities or the involvement affects both the upper and lower extremities. The upper extremities includes the arm, the forearm and the hand, including any joints connecting them. The upper extremities can also include bony or cartilaginous structures and joints above the waist. The lower extremities includes bones of the thighs, legs, feet and the patella (kneecap) including any joints connecting them. The lower extremities can also include bony or cartilaginous structures and joints connecting below the waist.

Clinical parameters of the present invention can be determined by any suitable means known or later developed. In one embodiment, clinical parameters can be determined by having a patient or healthcare professional answer a questionnaire related to the parameters. For example, patients can be asked to record the duration of their morning stiffness (in minutes). In addition, a 44-joint count for tender and swollen joint can be performed, where each joint is scored from a scale of 0-1. (See, van Riel et al., 2000; In: “EULAR handbook of clinical assessments in rheumatoid arthritis.”; Alphen aan den Rijn, The Netherlands: Van Zuiden Communications; 2000, 10-11). Other validated instruments for scoring clinical symptoms of RA or other forms of arthritis can be used, including without any limitation physician assessment of disease activity, 100 mm VAS, patient's global assessment of health 100 mm VAS, DAS 28, DAS 44, HAQ, HAQ or D1.

According to the present invention, the presence or absence of anti-MCV and additional clinical markers, the level of anti-MCV and additional clinical markers, as well as the determination or characteristics of clinical parameters can be used independently or in combination to assess the risk of developing RA from UA.

In one embodiment, the level and/or the presence or absence of anti-MCV or one or more clinical markers as well as the determination of clinical parameters are used in combination to assess the risk of developing RA from UA.

In another embodiment, the presence or absence of anti-MCV and duration of morning stiffness are used in combination to assess the risk of developing RA from UA. In yet another embodiment, the presence or absence of anti-MCV, duration of morning stiffness, age, gender, distribution of involved joints, number of tender joints, and number of swollen joints and any combinations thereof are used to assess the risk of developing RA from UA.

In yet another embodiment, the presence or absence of anti-MCV, anti-CCP, RF autoantibodies, as well as the level of CRP and/or ESR including any combinations thereof are used to assess the risk of developing RA from UA. In yet another embodiment, the present or absence of anti-MCV, anti-CCP, RF autoantibodies, as well as the level of CRP and/or ESR including any combinations thereof are used in combination with the determination of one or more clinical parameters to assess the risk of developing RA from UA.

In still another embodiment, the presence or absence of anti-MCV, anti-CCP, as well as the level of CRP and/or ESR are used either alone or in combination with the determination of one or more clinical parameters to assess the risk of developing RA from UA.

In another embodiment, the presence or absence of anti-MCV, anti-CCP, RF autoantibodies as well as the level of CRP and/or ESR are used in combination with the determination of one or more clinical parameters including age, gender, distribution of involved joints, duration of morning stiffness and combinations thereof to assess the risk of developing RA from UA.

In yet another embodiment, the presence or absence of anti-MCV, RF autoantibodies and anti-CCP are used in combination with the determination of one or more clinical parameters including age, gender, distribution of involved joints, duration of morning stiffness and combinations thereof to assess the risk of developing RA from UA.

In still another embodiment, the presence or absence of anti-MCV, RF autoantibodies, anti-CCP as well as the level of CRP and/or ESR are used in combination with the determination of one or more clinical parameters including age, gender, distribution of involved joints, duration of morning stiffness, number of tender joints and number of swollen joints and combinations thereof to assess the risk of developing RA from UA.

In still another embodiment, the presence or absence of anti-MCV and RF autoantibodies as well as the level of CRP and/or ESR are used in combination with the determination of one or more clinical parameters including age, gender, distribution of involved joints, duration of morning stiffness, number of tender joints and number of swollen joints and combinations thereof to assess the risk of developing RA from UA.

In still another embodiment, the presence or absence of anti-MCV and anti-CCP as well as the level of CRP and/or ESR are used in combination with the determination of one or more clinical parameters including age, gender, distribution of involved joints, duration of morning stiffness, number of tender joints and number of swollen joints and combinations thereof to assess the risk of developing RA from UA.

In still another embodiment, the presence or absence of anti-MCV, RF autoantibodies, and anti-CCP as well as the level of CRP and/or ESR are used in combination with the determination of one or more clinical parameters including age, gender, distribution of involved joints, duration of morning stiffness and combinations thereof to assess the risk of developing RA from UA.

In still another embodiment, the presence or absence of anti-MCV, RF autoantibodies, and anti-CCP are used in combination with the determination of one or more clinical parameters including age, gender, distribution of involved joints, duration of morning stiffness and combinations thereof to assess the risk of developing RA from UA.

In still another embodiment, the presence or absence of anti-MCV and anti-CCP as well as the level of CRP and/or ESR are used in combination with the determination of one or more clinical parameters including age, gender, distribution of involved joints, duration of morning stiffness and combinations thereof to assess the risk of developing RA from UA.

In still another embodiment, the presence or absence of anti-MCV and anti-CCP are used in combination with the determination of one or more clinical parameters including age, gender, distribution of involved joints, duration of morning stiffness and combinations thereof to assess the risk of developing RA from UA.

According to the present invention, when anti-MCV, one or more clinical markers as well as clinical parameters are used in combination for assessing the risk of developing RA from UA, one can assign certain risk values to these factors based on the characteristics or values of these factors. In general, one can develop various algorithms to evaluate anti-MCV as well as additional clinical markers or parameters in terms of their contributions to the risk of developing RA from UA. In one embodiment, the algorithm is based on a risk value assigned to each clinical marker or parameter and then evaluate the risk based on an entire collection of the relevant risk values. In another embodiment, the algorithm is based on a sum of risk values for a group of relevant clinical markers and/or clinical parameters.

According to the present invention, the risk value for each clinical marker or parameter can be assigned based on a predetermined risk value index or standard risk value. In other words, one can develop or pre-determine how much each clinical marker or parameter correlates with the risk of developing RA, e.g., by determining the percentage of RA development in patients positive of certain clinical markers and/or parameters or establishing regression coefficient values for each clinical marker or parameter. In addition, one can also develop or pre-determine the correlation, e.g., regression coefficient value between certain level, range or characteristics of clinical markers or parameters and assign a risk value index or standard risk value for such level, range and/or characteristics. Such predetermined risk value index or standard risk value can be used as a reference for assigning risk values for each relevant clinical marker and parameter. For example, certain risk value is associated with a range of certain level of a clinical marker, the presence or absence of one or more clinical markers, or the actual state or characteristics of a clinical parameter.

In one embodiment, one can assign risk values for clinical markers and parameters based on their regression coefficient values. In another embodiment, one can assign risk values for clinical markers and parameters based on normalized or mathematically manipulated correlation values for these markers and parameters. In yet another embodiment, one can assign risk values for combinations of two or more clinical markers and parameters, e.g., based on the regression coefficient values of each clinical marker and parameter. For example, one can assign a risk value for the categorical presence or absence of two or more clinical markers such as a respective risk value for the presence of either anti-MCV antibodies, or anti-CCP antibodies, or both anti-MCV antibodies and anti-CCP antibodies. In another exemplary embodiment, one can assign a risk value for the categorical characteristics of clinical parameters, e.g., localization categorical for tender and/or swollen joints.

In yet another embodiment, the risk value assigned for each of the clinical markers and parameters are shown in Table 1 below. For example, risk value is (1) 0.03 for each year of age; (2) 0 for the male gender or 0.5 for the female gender; (3) 0.5 in case where small joints in hands and feet, symmetric or upper extremities are involved, and 1 in case where both upper and lower extremities are involved; (4) 0.5 in case where the duration of morning stiffness is between about 30 minutes to about 59 minutes and 1 in case where the duration of morning stiffness is about 60 minutes or more; (5) 0.5 for 4-10 tender joints and 1 for 11 or more joints; (6) 0.5 for 4-10 swollen joints and 1 for 11 or more joints; (7) 0.5 for levels of CRP of 5-50 mg/L and 1 for levels 51 mg/L or more; (8) 0 for absence of RF autoantibody and 0 for presence of RF autoantibody; and (9) 0 for the absence of anti-MCV antibody or anti-CCP antibody, 1 for the presence of anti-MCV or anti-CCP antibody and 2.5 for the presence of anti-MCV antibody and anti-CCP antibody.

In another exemplary embodiment, risk value is (1) 0.02 for each year of age; (2) 0 for the male gender or 1 for the female gender; (3) 0.5 in case where small joints in hands and feet, symmetric and 1.5 in case where either upper or both upper and lower extremities are involved; (4) 0.5 in case where the duration of morning stiffness is between about 30 minutes to about 59 minutes and 1 in case where the duration of morning stiffness is about 60 minutes or more; (5) 0.5 for 4-10 tender joints and 1 for 11 or more joints; (6) 0.5 for 4-10 swollen joints and 1 for 11 or more joints; (7) 0.5 for levels of CRP of 5-50 mg/L and 1 for levels 51 mg/L or more; (8) 0 for absence of RF autoantibody and 1 for presence of RF autoantibody; and (9) 0 for the absence of anti-MCV antibody or anti-CCP antibody, 1 for the presence of anti-MCV and 2 for the presence of anti-CCP antibody.

TABLE 1
Assigned Risk Value for Various Markers and Parameters

| | Regression | | | |
|---|---|---|---|---|
| Parameters | Coefficient | Original | Rederived | Enhanced |
| (Parameter State or Values) | Values | Risk Value¹ | Risk Value | Risk Value |
| C-Reactive Protein: | | | | |
| 5 mg/L | 0 | 0 | 0 | 0 |
| 5-50 mg/L | 0.6 | 0.5 | 0.5 | 0.5 |
| >50 mg/mL | 1.6 | 1.5 | 1.5 | 1.5 |
| Rheumatoid Factor: | | | | |
| Absence | 0 | 0 | 0 | 0 |
| Presence | 0.8 | 1 | 1 | 0 |
| Anti-CCP2 Ab | | | | See |
| Absence | 0 | 0 | 0 | combination of |
| Presence | 2.1 | 2 | 2 | anti-MCV and |
| | | | | CCP3 Ab |
| Anti-MCV Ab: | | | | See |
| <20 U/Ml | nd² | nd | nd | combination of |
| >20 U/mL OR | nd | nd | nd | anti-MCV and |
| | | | | CCP3 Ab |
| Either anti-MCV or CCP3 Ab | nd | nd | nd | 1 |
| Both anti-MCV and CCP3 Ab | nd | nd | nd | 2.5 |
| Each year of Age (max. 100 years): | 0.02 | 0.02 | 0.02 | 0.03 |
| Gender: | | | | |
| Male | 0 | 0 | 0 | 0 |
| Female | 0.8 | 1 | 1 | 0.5 |
| Distribution of Involved Joints: | | | | |
| small joints of hands and feet | 0.6 | 0.5 | 0.5 | 0.5 |
| symmetrical involvement | 0.5 | 0.5 | 0.5 | 0.5 |
| upper extremities or | 0.8 | 1 | 1 | 0.5 |
| upper and lower extremities | 1.3 | 1.5 | 1.5 | 1 |
| Morning Stiffness: | | | | |
| Length of VAS <26 mm | 0 | 0 | nd | nd |
| Length of VAS 26-90 mm | 1 | 1 | nd | nd |
| Length of VAS >90 mm | 2.2 | 2 | nd | nd |
| Length of time 30-59 min | nd | nd | 0.5 | 0.5 |
| Length of time ≧60 min | nd | nd | 1 | 1 |
| Number of Tender Joints: | | | | |
| 4-10 | 0.6 | 0.5 | 0.5 | 0.5 |
| >10 | 1.2 | 1 | 1 | 1 |
| Number of Swollen Joints: | | | | |
| 4-10 | 0.4 | 0.5 | 0.5 | 0.5 |
| >10 | 1 | 1 | 1 | 1 |

¹Simplified, rounded values of original regression coefficient values
²nd = not determined

Of course, one skilled in the art would understand that the absolute value provided here should not be limiting as along as the relative risk value (or the ratios) among all the relevant clinical markers and parameters are maintained the same as the ones listed in these exemplary embodiments.

In yet another aspect, the invention for determining a predicted risk of an individual with UA developing RA includes a system having a blood analyzer and a computing device. The blood sample analyzer is configured for analysis of the blood sample from one or more individual in order to determine the presence or absence, or levels of at least one or more clinical markers such as but not limited to anti-MCV antibodies, anti-CCP antibodies, RF autoantibodies, CRP, HS-CRP and/or ESR. The anti-CCP antibodies can be directed to CCP1, CCP2 and/or CCP3. In one embodiment, the blood analyzer is configured to determined the levels of at least one or more of the above clinical markers. The computing device for the system in the invention can also be configured to assign a risk value to each of the clinical marker determined by the blood sample analyzer. The risk value assigned can be based on predefined risk values associated with each of the clinical marker that is stored in the memory. Based on the collection of risk values assigned to the clinical markers, the computing device then determines a predicted risk of the individual developing RA.

In a further aspect, the invention provides a combination of tests useful for predicting whether an individual with UA will develop RA. The combination of tests comprise testing for the presence or absence of anti-MCV antibodies, RF autoantibodies, anti-CCP antibodies, serum levels of CRP, HS-CRP, or ESR. The combination of tests can include testing for levels of anti-MCV antibodies, RF autoantibodies, anti-CCP antibodies, serum levels of CRP, HS-CRP, or ESR. In one embodiment, the combination of tests comprise a first test for the presence or absence of anti-MCV antibodies and a second test where the second test can be a test for the serum level of CRP, HS-CRP, ESR or test for the presence or absence of RF autoantibody or anti-CCP antibody. In another embodiment, the combination of tests comprise a first test, a second test and a third test where the first test is for the presence or absence of anti-MCV antibodies or levels thereof, the second test is for the serum level of CRP, HS-CRP or ESR and the third test is for the presence or absence of RF autoantibodies or anti-CCP antibodies or levels thereof. In a further embodiment, the combination of tests comprises a first test, a second test, a third test and a fourth test where the first test is for the presence or absence of anti-MCV antibodies or levels thereof, the second test is for the serum level of CRP, HS-CRP or ESR, the third test is for the presence or absence of RF autoantibodies or levels thereof and the fourth test is for the presence or absence of anti-CCP antibodies or levels thereof. In still another embodiment, the combination of tests include a combination of rapid lateral flow tests for the detection of anti-MCV, RF autoantibodies, and optionally anti-CCP antibodies. Such combination can be provided in a single rapid lateral flow test or one or more lateral flow tests.

The combination of tests of the invention where the test for the presence or absence of anti-MCV antibody is to be determined can be carried out using one or more peptides derived from native vimentin or variants thereof. The peptide used in the combinations tests for the presence or absence of anti-MCV antibody can be of varying lengths. The peptide length can be from between about 3 amino acids to about 10 amino acids, from between about 10 amino acids to about 50 amino acids, from between about 50 to about 100 amino acids, from between about 100 amino acids to about 200 amino acids, from between about 200 amino acids to about 300 amino acids, from between about 300 amino acids to about 400 amino acids, from between about 400 amino acids to about 500 amino acids. In one embodiment, the amino acid sequence of peptide used in the combination test can be about 10%, about 20%, about 30%, about 40%, about 50%, about 60%, about 70%, about 80%, about 90% or about 100% identical to native vimentin or variants thereof.

Variants can include native vimentin having one or more additional amino acids in the protein sequence. The additional amino acid can be arginine leucine, proline, threonine, tyrosine, lysine, histidine, alanine, cysteine, aspartic acid, glutamic acid, phenylalanine, glycine, isoleucine, methionine, asparagine, glutamine, serine, valine, trytophan residue or combination thereof, which can be D- or L-amino acids. The additional amino acid in the sequence can also be a post-translationally modified amino acid, for example, the additional amino acid in the native vimentin sequence can be a citrulline. Accordingly, in certain embodiments, the presence or absence of anti-MCV antibodies in the combination tests can include using a peptide or fragment of the polypeptide derived from native vimentin having at least one additional arginine residue.

In certain embodiment, the peptide or fragment to be included in the combination tests for detecting anti-MCV antibodies can have one or more additional arginine residue in at least one of positions 16, 17, 19, 41, 58, 59, 60, 68, 76, 140, 142, 147, 363, 406 or 452. In certain other embodiments, at least one arginine in the form of citrulline, can be, for example, in at least one of positions 4, 12, 23, 28, 36, 45, 50, 64, 71, 100, 320, 364 or 378. In other embodiments, the preferred position can be at least be one of positions 41, 58, 59 and/or 60. In another embodiment, the peptide or fragment can have two, three or more unmodified arginines or citrulline or combination thereof in any one of the amino acid positions recited above.

In other embodiments, the peptide or fragment to be included in the combination tests for detecting anti-MCV antibodies can have one or more additional leucine residue in at least one of positions 3, 20, 33, 36, 37, 94, 165, 361, 399 or 426, preferably in positions 33, 36 and/or 37 of the mutated citrullinated vimentin or native vimentin. In another example, the peptide or fragment can have one or more an additional proline residue in at least one of positions 21, 41, 43, 50, 54, 62, 64 or 89, preferably in positions 41, 43, 50, 54, 62 and/or 64 of the mutated citrullinated vimentin or native vimentin. In yet another example, the peptide or fragment can have one or more an additional threonine residue can be in at least one of positions 24, 35 or 99. In a further example, the peptide or fragment can have one or more an additional tyrosine residue in at least one of positions 25, 39, 42, 49, 55 or 67. In certain embodiments can have two, three or more arginine, citrulline, leucine, proline, threonine, or tyrosine or combination thereof.

In a further aspect, the invention provides a combination of tests useful for predicting whether an individual with UA will develop RA wherein the combination of tests comprise testing for the presence or absence of MCV or native vimentin protein fragments or peptides, RF autoantibodies, anti-CCP antibodies, serum levels of CRP, HS-CRP, or ESR. The combination of tests can include testing for levels of MCV or native vimentin protein fragments or peptides, RF autoantibodies, anti-CCP antibodies, serum levels of CRP, HS-CRP, or ESR. In one embodiment, the combination of tests comprise a first test for the presence or absence of MCV or native vimentin protein fragments or peptides and a second tests where the second test can be tests for the serum level of CRP, HS-CRP, ESR or tests for the presence or absence of RF autoantibody or anti-CCP antibody. In another embodiment, the combination of tests comprise a first test, a second test and a third test where the first test is for the presence or absence of anti-MCV antibodies or levels thereof, the second test is for the serum level of CRP, HS-CRP or ESR and the third test is for the presence or absence of RF autoantibodies or anti-CCP antibodies or levels thereof. In a further embodiment, the combination of tests a first test, a second test, a third test and a fourth test where the first test is for the presence or absence of anti-MCV antibodies or levels thereof, the second test is for the serum level of CRP, HS-CRP or ESR, the third test is for the presence or absence of RF autoantibodies or levels thereof and the fourth test is for the presence or absence of anti-CCP antibodies or levels thereof.

In some embodiments, the combination tests comprises testing for the presence or absence of nucleic acids or polynucleotides such as DNA, RNA or fragments thereof encoding vimentin, RF, CCP, CRP and/or or variants thereof.

In another aspect, the invention provides a method of providing useful information for predicting whether an individual with UA will develop RA. The method includes determining a set of clinical markers for the individual and providing the set of clinical markers to an entity that combines the set of clinical markers with a set of clinical parameters to provide the prediction. The set of clinical markers to be determined can include the presence or absence of anti-MCV antibodies or MCV peptides or fragments thereof, and at least one clinical marker, such as but not limited to the serum level of CRP, HS-CRP or ESR, the presence or absence of RF autoantibody, and the presence or absence of anti-CCP antibodies. The set of clinical parameters include the duration of morning stiffness of the individual. In certain embodiments, the set of clinical parameters include at least two clinical parameters, for example, the duration of morning stiffness of the individual, the age of the individual, the gender of the individual, the localization of the joint complaints of the individual, the number of tender joints of the individual, and the number of swollen joints of the individual. The entity receiving such information can be a point of care provider such as a clinician, nurse, a hospital or clinic, a hospital database, a data processing center, a webpage address, a patient, an internet address set up for a patient or clinician, etc.

In another aspect, the invention provides a collection of results that is useful for predicting whether an individual with UA will develop RA. The collection of results include values for a set of clinical markers for the individual. In one embodiment the collection of results include a first ser of clinical markers such as the presence or absence of anti-MCV antibodies or MCV peptides or fragments thereof and at least one additional clinical marker, for example, the serum level of CRP, HS-CRP or ESR, the presence or absence of RF autoantibody, and the presence or absence of anti-CCP antibodies. In another embodiment, the collection of results comprises a first set of clinical markers such as the presence or absence of anti-MCV antibodies or MCV peptides or fragments thereof, the serum level of CRP, HS-CRP or ESR, the presence or absence of RF autoantibody, and the presence or absence of anti-CCP antibodies. In certain other embodiments, the collection of results include instruction for using the values for the first set of clinical markers in combination with a set of other clinical parameters. The set of other clinical parameters include one or more of the following clinical parameters such as but not limited to the duration of morning stiffness of the individual, the age of the individual, the gender of the individual, the localization of the joint complaints of the individual, the number of tender joints of the individual, and the number of swollen joints of the individual. Such collection can be provided in any suitable form, e.g., hard copy paper record, electronic copy or transmission, etc.

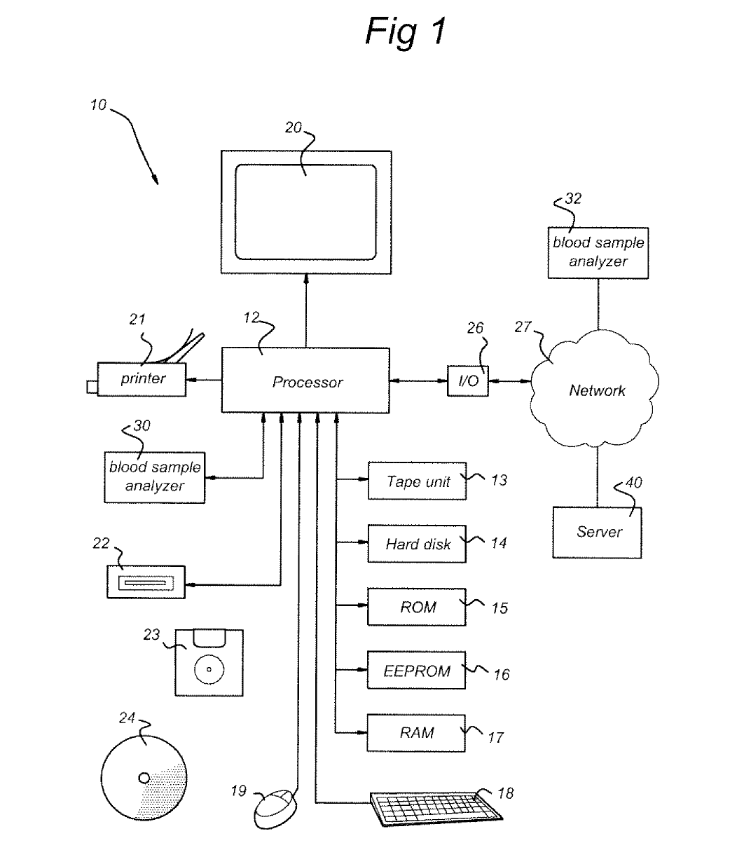

In one aspect, the invention provides a computer having a processor and a memory, where the processor is arranged to read from the memory and write into the memory. The schematic in FIG. 1 described in Example 1 below shows the relationship between the computer hardware used for one or more embodiments described herein for predicting the risk of an individual with UA developing RA. In one embodiment the computer can be personal computers, servers, laptops, personal digital assistance (PDA), palmtops, cell phones and devices capable of transmitting and receiving data. The memory stores instructions and data, for example, data of the presence or absence or levels of one or more clinical markers, data derived from the clinical parameter values, etc. The memory may also comprise program lines readable and executable by the processor. The program lines provides the computer with the functionality to perform one of the methods for predicting the risk that an individual with UA will develop RA described herein. Examples of memory include a tape unit, hard disk, a Read Only Memory (ROM), Electrically Erasable Programmable Read Only Memory (EEPROM) and/or a Random Access Memory (RAM). Data and instructions are arranged in the memory of the computer in such manner as to provide the processor with the capacity to perform mathematical operations used for predicting whether an individual with UA will develop RA. Thus in one embodiment, the memory comprises data and instructions arranged to provide the processor with the capacity to perform of method of predicting whether an individual with UA will develop RA. In another embodiment the computer system comprises program lines readable and executable by the processor. Further, the processor can be connected to one or more input devices, such as a keyboard, a mouse; one or more output devices, such as a display and a printer; and one or more reading units to read, e.g., the floppy disks or CD-ROM.

In another embodiment, the computer is connected to an input/output device such as a sample analyser for analysing body fluid samples, e.g. blood samples or other biological samples by performing measurements on the samples. The sample analyzer can be located proximate with the computer and/or remotely from the computer, where communication with the computer is via a communication network through direct wired and/or wireless communication. In one embodiment, a number of analyzers can be in communication with the computer. In other embodiments, multiple sample analyzers can be in communication remotely with the computer. The analysis data signals obtained from the sample analyzer are received by or inputted into the computer in a manner that provides the processor with the capacity to determine from the analysis data signals: i) the serum level of C-reactive protein; ii) the presence or absence of RF autoantibody; iii) the presence or absence of anti-CCP antibodies; and iv) the presence or absence of anti-MCV antibodies or MCV peptides or fragments thereof present in said sample as clinical parameters. The processor may be arranged for calculating a prediction score based on the sum of the risk values for each parameter value. Alternatively, the processor is arranged for determining the predicted risk for the individual on developing rheumatoid arthritis by correlating the prediction score for the individual with the risk associated with that prediction score in accordance with a predetermined probability distribution as described herein above. Accordingly, the computer may be arranged to read at least one clinical parameter and/or clinical marker as determined by the sample analyser and stored in the memory units. The computer may also determine at least one clinical parameter by reading from the memory, or from input devices, such as keyboard and mouse, or from one or more reading units to read for instance floppy disks or CD ROM.

The computer may further be arranged to receive a set of further clinical parameter values comprising the duration of morning stiffness; the age of the patient; the gender of the patient; the localization of the joint complaints; the number of tender joints involved; and the number of swollen joints involved. In other embodiments, fewer or additional further clinical parameters values may be received by the computer and used in developing a predicted risk of the individual with UA progressing to RA. In one embodiment, for example, the further clinical parameter values are entered into the computer using one or more input devices, such as a keyboard and/or a mouse in response to information displayed in a graphical user interface that is displayed on the display device. For example, a graphical user interface may be configured to prompt a user to enter each of a plurality of clinical parameter values. In one embodiment, each of the entered clinical parameter values are used to determine a predicted risk of developing rheumatoid arthritis. In other embodiments, selected clinical parameter values are used in determining a predicted risk of developing rheumatoid arthritis (referred to herein as a “predicted risk”). In one embodiment, a confidence level in the predicted risk increases as the number of clinical parameter values that are entered into the graphical user interface and processed by the computer increases. Thus, while a predicted risk may be determined based on as few as two clinical parameter values, the confidence level of the predicted risk may increase as additional clinical parameter values are received and considered in developing the predicted risk.

In one embodiment, the computer may be arranged to read these further parameter values from memory, from input devices, such as keyboard and mouse, or from one or more reading units to read for instance floppy disks or CD ROM's.

Further, the computer may be arranged to determine a predicted risk of the individual developing rheumatoid arthritis by correlating at least two of the clinical parameter values with a predefined risk value associated with each particular parameter value. The predicted risk score may be outputted by the computer using one or more output devices, such as display and printer. Also, computer may be arranged for transmission of the predicted risk value over the network to another computer system (not shown).

In one embodiment the predicted risk is transmitted to a remote computing system and displayed to a user via a graphical user interface. In another embodiment, the predicted risk is transmitted via e-mail to the individual, a physician, and/or another computing system. In yet another embodiment, the predicted risk may be transmitted via facsimile or printed and delivered to the individual and/or physician. In certain embodiments, the risk values associated with each of the clinical parameter values and the total risk value for the individual are also transmitted from the computer to another computing device. In one embodiment, the predicated risk is stored on the server and is accessible to users with proper authorization to view the predicted risk, such as the individual and the individual's healthcare providers.

In another aspect, the invention provides a system for determining a predicted risk of an individual with UA to develop RA. The system comprises means for receiving at least one or more characteristic clinical parameter, and means for receiving at least one or more additional characteristic clinical marker. For example, the clinical parameter, includes but is not limited to the age, the gender, the distribution of involved joints, the duration of morning stiffness, the number of tender joints, and the number of swollen joints. Non-limiting examples of clinical markers such as but not limited to anti-MCV antibody, anti-CCP antibody, RF autoantibody, CRP, HS-CRP, ESR or a combination thereof. In one embodiment the system comprises a means for receiving a characteristic of a first clinical marker comprising anti-MCV antibody and optionally a second clinical marker. Non-limiting examples of a second clinical marker includes anti-CCP antibody, RF autoantibody, CRP, HS CRP or ESR. The system further comprises a means for assigning a risk value to each of the clinical parameter and clinical marker characteristic received, and a means for determining a predicted risk of the individual developing RA based at least partly on the assigned risk values.

### EXAMPLES

### Example 1

### Schematic of a Computer for Performing the Method of Predicting Risk of Developing RA in a Patient with UA

FIG. 1 shows a schematic example of an embodiment of a computer 10 as may be used in one or more of the embodiments described herein. As illustrated in exemplary FIG. 1, the computer 10 comprises a processor 12 for performing arithmetical operations. The processor 12 is connected to memory units that may store instructions and data, such as a tape unit 13, hard disk 14, a Read Only Memory (ROM) 15, Electrically Erasable Programmable Read Only Memory (EEPROM) 16 and a Random Access Memory (RAM) 17. The processor 12 is also connected to one or more input devices, such as a keyboard 18 and a mouse 19, one or more output devices, such as a display 20 and a printer 21, and one or more reading units 22 to read for instance floppy disks 23 or CD ROM's 24.

The computer 10 shown in FIG. 1 may also comprise an input output device (I/O) 26 arranged to communicate with other computer systems (not shown) via a communication network 27. The sample analyser is in data communication with the network 27, and is positioned either locally proximate 30 and/or remotely positioned 32 from the computer.

A server 40, which stores data received from the sample analyzer 30, 32 and provides the data to the computer 10, is also in data communication with the network 27 via a graphical user interface. The server 40 stores data received from the sample analyser 30, 32 and provides this data to the computer 10. The server 40 and/or the sample analyser 30, 32 can be configured to perform operations on data determined by the sample analyser 30, 32 in order to determine a predicted risk of an individual developing rheumatoid arthritis, such as by using the systems and methods described above. The predicted risk score may be outputted by the computer 10 using one or more output devices, such as display 20 and printer 21, or transmitted over network 27 to another computer system (not shown). The predicted risk score can be transmitted to the individual and/or a physician via e-mail, facsimile to another computer, PDA, cell phone or other electronic devices, printed and delivered or stored on the server 40 for access by users with proper authorization to view the predicted risk, such as the individual or the individual's health care provider.

### Example 2

### Schematic Depiction of a Flow Diagram of a Procedure Executed by a Computer According to an Embodiment of the Invention

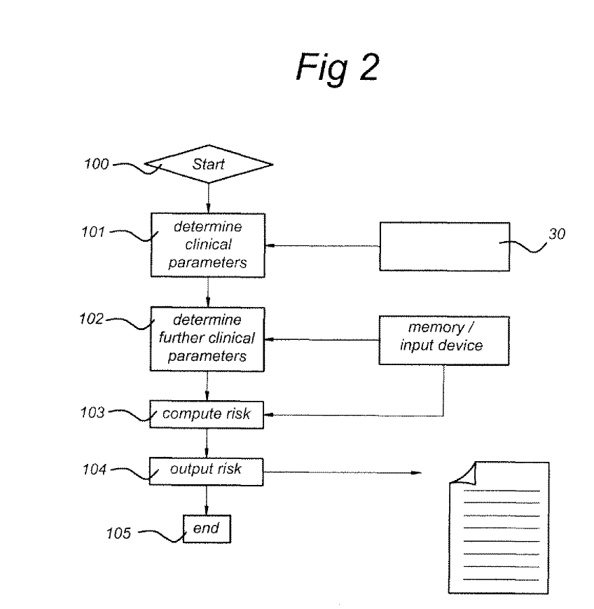

FIG. 2 schematically depicts a flow diagram of a procedure as may be executed by computer 10, or other computing devices, according to an embodiment of the invention. Depending on the embodiment, certain of the actions described below may be removed, others may be added, and the sequence of actions may be altered. The following description refers to FIG. 2 and FIG. 1 for specific hardware involved in the procedure of FIG. 2

In a first action 100, the computer 10 starts executing the procedure. The execution of the procedure can be triggered by input from a user into a graphical user interface displayed on the display device 20. In a next action 101, the computer 10 determines at least one clinical parameter using sample analyser 30, 32, in, for example, the following steps: (a) the processor 12 requests the sample analyser 30, 32 to output data-signals relating to the measured values of a blood sample to the processor 12, where the output data-signals comprise parameter values associated with each of one or more clinical parameters, such as, for example, a parameter value indicating a serum level of C-reactive protein in the blood sample and a parameter value indicating presence or absence of RF in the blood sample; (b) the processor 12 receives the data signals and (c) the processor optionally stores the data-signals relating to the measured values in memory 13, 14, 15, 16, 17 of FIG. 1. Step (a) may also comprise that the processor 12 requests the sample analyser 30, 32 perform certain measurements on the blood sample relating to determining a set of clinical parameter values, such as clinical parameters values for clinical parameters before transmitting the data-signals.

In a next action 102, the processor 12 determines at least one of the further clinical parameter values using one or more input devices as described above, or alternatively, from associated data already stored in memory 13, 14, 15, 16, 17. Alternatively, the further clinical parameter values may be entered into a computing device, such as computer 10, via a graphical user interface or by a caregiver in response to comments from the individual. The further clinical parameter values can also be entered by the individual if a user interface is made accessible to the individual via a computer in communication with the network.

In a further action 103, the computer 10 determines a predicted risk of an individual developing rheumatoid arthritis by correlating each of at least two of the clinical parameter values and further clinical parameter values determined in action 101 and 102 above with predefined risk values that are associated with each particular parameter value. These risk values may then be combined in order to determine a total risk value for the individual. Finally, the total risk value may be associated with a predicted risk of the individual developing rheumatoid arthritis. In addition, ranges of values for each of the clinical parameter values can be used to associate with particular risk values. Risk values for particular clinical parameters can also be determined according to formulas specific to each clinical parameter. The total risk value is the sum of each of the risk values that have been associated with the clinical parameter values. Alternatively, the total risk value may be calculated using only a portion of the risk values.

In a next action 104, the computer 10 outputs the computed predicted risk of an individual of developing rheumatoid arthritis by using one or more output devices, such as display 20 and printer 21 or by transmission of the computed predicted risk to another computer system (not shown), such as via email or storage of the predicted risk on a server that is accessible to other users. Also, the computer 10 may store the computed predicted risk, and/or the risk values and total risk values, in memory 13, 14, 15, 16, 17 or on the server 40.

In action 105, the execution of procedure ends. If needed, the procedure may be resumed at action 101 to execute once more.

### Example 3

### Table Illustrating Exemplary Risk Values that are Associated with Ranges of Parameter Values for Several Clinical Parameters

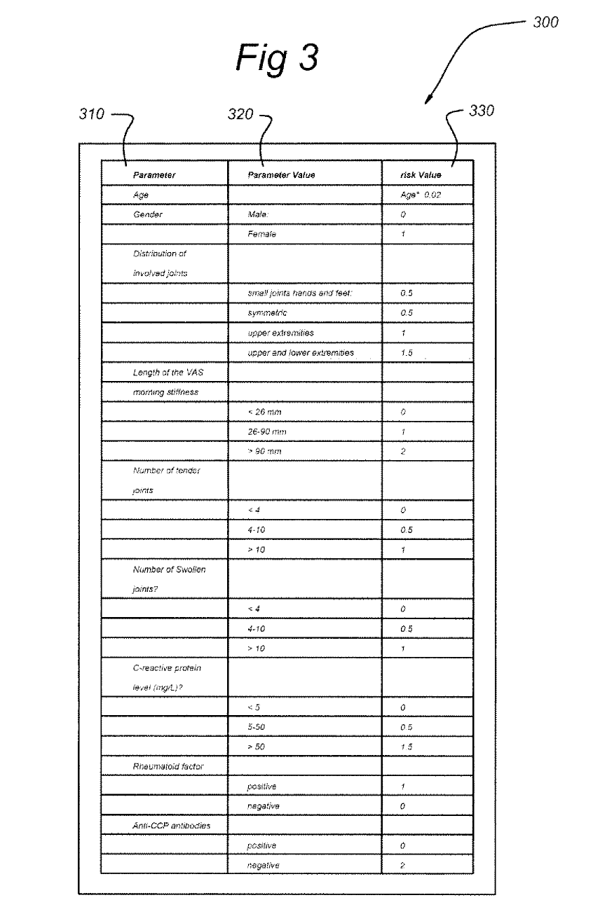

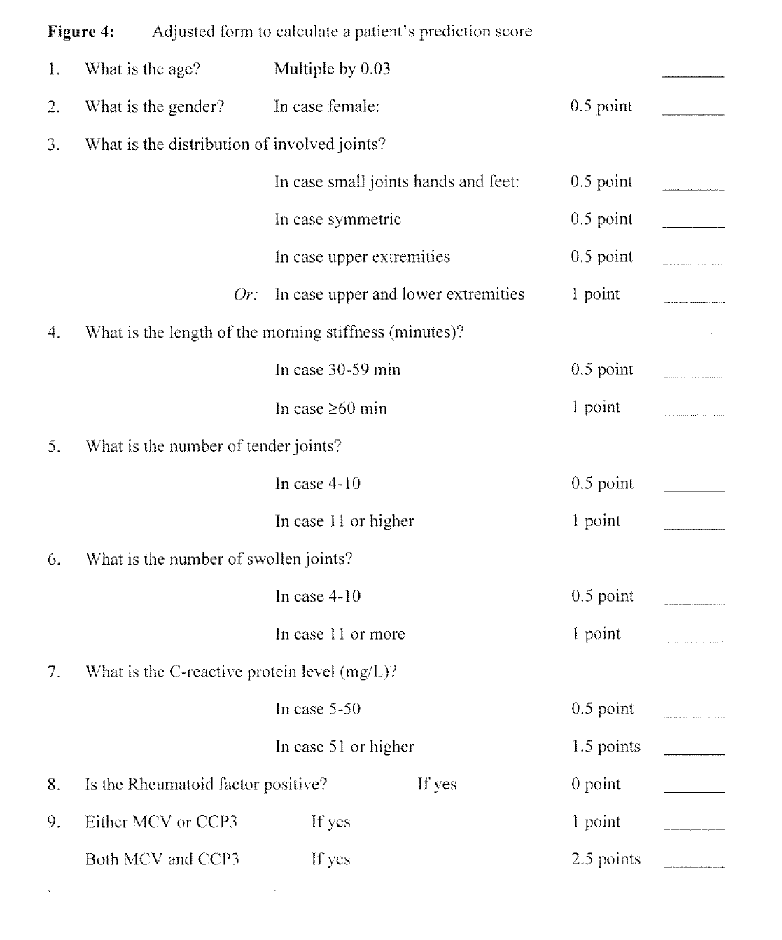

FIG. 3 is a table 300 illustrating exemplary risk values that are associated with ranges of parameter values for several clinical parameters. In the embodiment of FIG. 3, risk values are associated with each of nine clinical parameters. In other embodiments, fewer or more clinical parameters may be associated with risk values. The table 300 may advantageously be stored in a memory device and accessed by the computer 10 in order to determine risk values for any of the listed parameters. The table 300 may be stored in a memory of the computer 10, at the server 40, or at the sample analyser 30, 32. In another embodiment, the table 300 is converted to a worksheet format, such as will be discussed below with reference to FIG. 4, that may be printed or viewed in a graphical user interface.

In the embodiment of FIG. 3, a first column 310 lists clinical parameters, a second column 320 lists possible parameter values associated with each of the clinical parameters, and a third column 330 lists a risk value that is associated with respective ranges of parameter values.

In one embodiment, each of the risk values assigned to an individual are summed in order to determine a total risk value that will be associated with a predicted risk of the individual developing rheumatoid arthritis. Below are exemplary parameter values for two individuals, individual A and individual B, and the associated risk values assigned to the individuals using the table 300.

TABLE 2
Risk Values and Total Risk Value for Individual A

| Parameter | Parameter Value | Assigned Risk Value |
|---|---|---|
| Age | 50 | 1 (i.e., 50*.02) |
| Gender | Male | 0 |
| Distribution of involved | Upper and lower | 1.5 |
| joints | extremities | |
| Length of VAS morning | 56 mm | 1 |
| stiffness | | |
| Anti-MCV antibodies | Positive | 1 |
| Number of tender joints | 12 | 1 |
| Number of swollen joints | 7 | 0.5 |
| C-reactive protein level | 12 | 0.5 |
| Rheumatoid factor | Negative | 0 |
| Anti-CCP antibodies | Positive | 2 |
| Total Risk Value | | 8.5 |

TABLE 3
Risk Values and Total Risk Value for Individual B

| Parameter | Parameter Value | Assigned Risk Value |
|---|---|---|
| Age | 75 | 1.5 (i.e., 75*.02) |
| Gender | Female | 1 |
| Distribution of involved | Symmetric | 0.5 |
| joints | | |
| Anti-MCV antibodies | Positive | 1 |
| Number of tender joints | 12 | 1 |
| Number of swollen joints | 10 | 0.5 |
| C-reactive protein level | 52 | 1.5 |
| Rheumatoid factor | Positive | 1 |
| Anti-CCP antibodies | Positive | 2 |
| Total Risk Value | | 10 |

As indicated above, the total risk value for individual A is 8.5, while the total risk value for individual B is 10. In one embodiment, a higher total risk value indicates a higher risk of developing rheumatoid arthritis. Thus, in this embodiment, individual B is more likely to develop rheumatoid arthritis than individual A. In other embodiments, however, lower total risk scores may indicate lower risks of developing rheumatoid arthritis.

As described in further detail below, these total risk values may now each be associated with a corresponding predicted risk of the individual developing rheumatoid arthritis. In one embodiment, each of the parameter values for the individuals are entered into a computing device, such as the computer 10 via a graphical user interface, and the computing device determines the risk values associated with each of the parameter values such as by accessing table 300 stored in a memory. In the embodiment described below with respect to FIG. 4, a user manually selects the risk values associated with particular parameter values and calculates a total risk value.

### Example 4

### Checklist used to Record Clinical Parameter Values and Associated Risk Values with Each of the Clinical Parameter Values

FIG. 4a illustrates an exemplary checklist 400a that may be used to record clinical parameter values and associate risk values with each of the clinical parameter values. In the embodiment of FIG. 4, a user, such as a physician, records information regarding the patient on the checklist 400a, and assigns risk values to each of the parameter values associated with the particular parameter value. In FIGS. 3 and 4, specific parameters, as well as specific risk values associated with each of the parameters are used in determining the total risk value for the individual. However, fewer or more parameters may be used in order to determine a total risk value. Additionally, the risk values associated with parameter values may be higher or lower depending on the specific implementation. For example, only a portion of the parameters listed in FIG. 3 can be used and, the risk values associated with certain parameter values may be adjusted.

FIG. 4b illustrates another exemplary checklist 400b. In this exemplary checklist 400b, anti-MCV antibodies substitute for RF and morning stiffness duration is substituted for morning stiffness severity in checklist 400a. In addition, the risk values are adjusted as shown for calculation of patients prediction score.

### Example 5

### Graph illustrating a Predicted Risk of Developing RA as a Function of the Total Risk Value

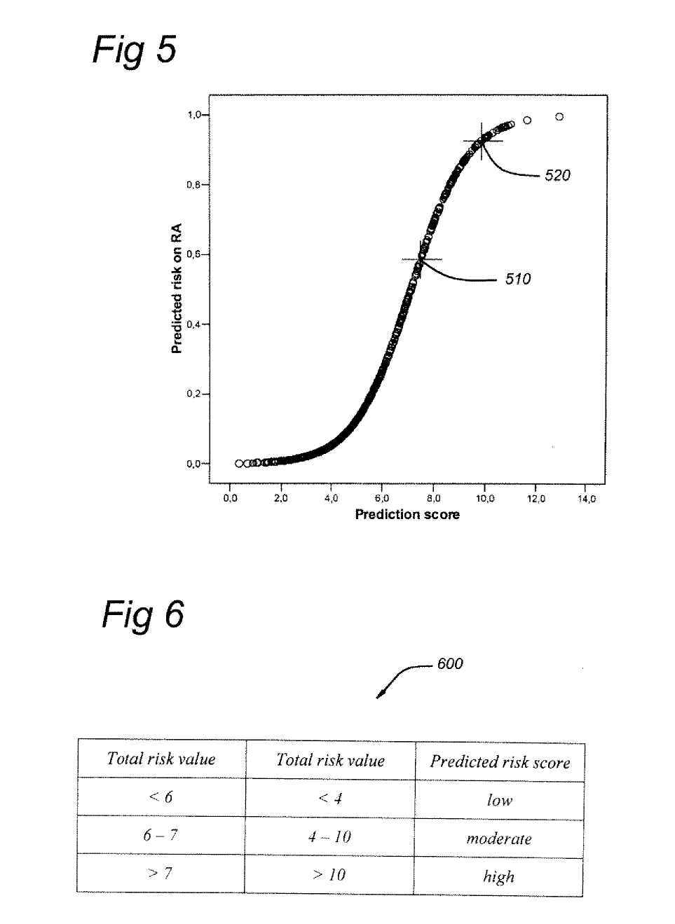

FIG. 5 is a graph illustrating a predicted risk of developing rheumatoid arthritis as a function of the total risk value. In the embodiment of FIG. 5, the vertical axis represents a predicted risk of an individual developing rheumatoid arthritis, while the horizontal axis represents an individual's total risk value (Prediction Score). Thus, a total risk value may be associated with a predicted risk using the graph of FIG. 5. For example, with regard to individual A shown in Table 2 above, a total risk value of 8.5 was calculated. Using the graph of FIG. 5, individual A may be assigned a percentage predicted risk. For example, a risk score of 60% (see intersection at about point 510) indicates that the individual has a 60% chance of developing rheumatoid arthritis. Using the graph of FIG. 5 again, individual B shown in Table 3 above was assigned a total risk value of 10, which corresponds with a predicted risk of about 90% (see intersection at about point 520). Thus, in this embodiment individual B has about a 90% risk of developing rheumatoid arthritis.

In one embodiment, predicted risk data, such as the data illustrated in FIG. 5, may be expressed as an algorithm that converts a total risk value to a predicted risk. In this embodiment, once a total risk value is determined, the algorithm may automatically convert the total risk value to a percentage predicted risk that the individual develops rheumatoid arthritis. In one embodiment, the algorithm calculates the predicted risk after each of the parameter values are entered into, or received by, the computer 10. In another embodiment, the computer 10 is configured to execute an algorithm to determine a predicted risk score after entry of each parameter value. Accordingly, a physician or user entering parameter values may watch the predicted risk change as additional parameter values are entered into the computer 10.

### Example 6

### Exemplary Table Storing Exemplary Total Risk Values Associated with Predicted Risk Scores

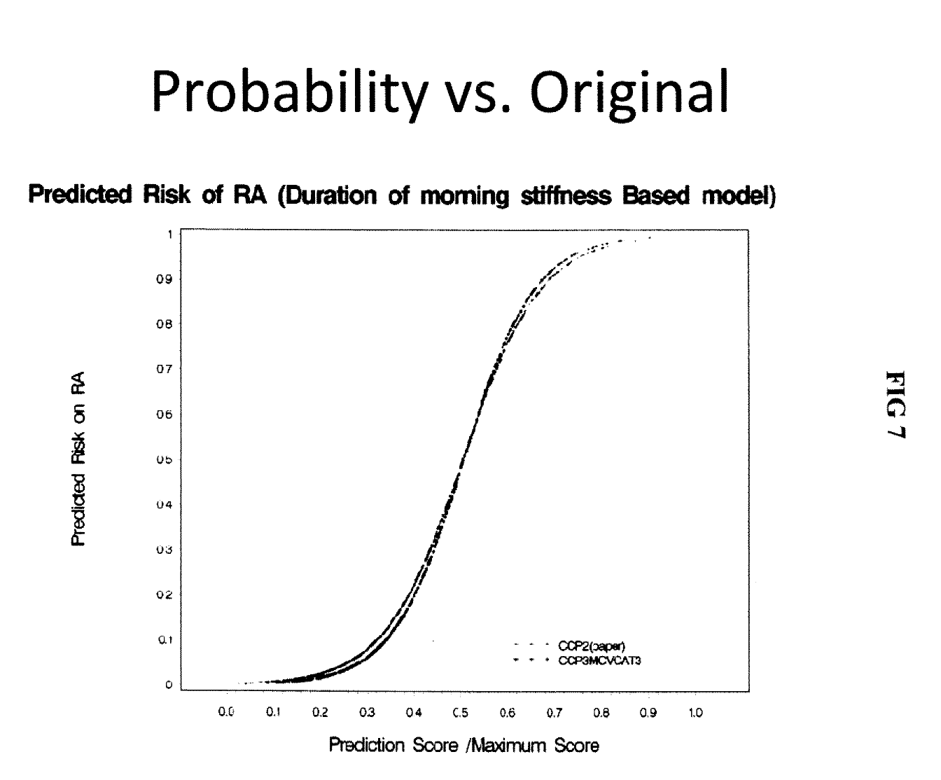

FIG. 6 illustrates a table 600 storing exemplary total risk values associated with predicted risk scores. In the embodiment of FIG. 6, a total risk value of less than four is associated with a predicted risk score of “low”, indicating that the individual has a low predicted risk of developing rheumatoid arthritis. In this embodiment, a total risk value of greater than 10 is associated with a predicted risk score of “high”, while total risk values in the range of 4-10 are associated with a predicted risk or of “moderate.”

The predicted risk scores illustrated are exemplary, and are not intended to limit the scope of predicted risk scores that may be used in conjunction with the systems and methods described herein. For example, in certain embodiments, the predicted risk scores may be numerical, such as percentages. In other embodiments, the predicted risk scores may be analogous to grades, such as giving the individual a grade from A-F, where A indicates a very low risk of developing rheumatoid arthritis and F indicates a very high risk of developing rheumatoid arthritis. In other embodiments any other type of predicted risk score may be associated with a total risk value and provided to an individual.

### Example 7

### Development of Specific Models for Associating Parameter Values with Risk Values and Associating Total Risk Scores with Predicted Risk Scores

The following is a discussion for the development of specific models for associating parameter values with risk values and associating total risk scores with appropriate predicted risk scores. The following clinical test data is provided as exemplary methods for generating such models, and is not intended as a limitation of other methodologies that may be used to develop similar models, or of the parameters, risk values, or predicted risk scores that may be used in a model.

A predicted risk score model was derived using three different cohorts of patients with recent-onset UA. (Discussed below under Validation Cohorts) In two of these cohorts, data on the baseline parameter morning stiffness severity measured on a Visual Analogue Scale (VAS) was not available, but the duration of morning stiffness (in minutes) was recorded. Therefore, the prediction rule was re-derived in the derivation cohort (Leiden Early Arthritis Clinic (EAC)) using the duration of morning stiffness as a substitute. The prediction rule in the Leiden cohort is described below and in copending, commonly owned U.S. patent application Ser. No. 11/697,665, the entire contents of which are incorporated herein by reference. The negative and positive predictive values, as well as the area under the receiver operator characteristic curve (AUC) of this adjusted model were assessed.

### Validation Cohorts

Patients from three separate cohorts who had an early UA were studied. The first cohort represents the UA-patients recruited to the Birmingham Early Arthritis cohort. This very early arthritis cohort recruits are diagnosed with synovitis in at least one joint and having a symptom duration (of inflammatory joint pain, swelling or morning stiffness) of ≦3 months. The cohort has been described in detail previously. (Raza et al., Arthritis Res Ther 2005; 7:R784-R795). Patients were followed for at least 18 months and patients were classified as having RA if they fulfilled the 1987 ACR-criteria for RA.

The second cohort are the patients included in the Berlin Early Arthritis Clinic; this clinical study started in January 2004 and patients were included if they had synovitis in at least two joints and a symptom duration of between 4 weeks and 12 months (Detert et al., Deutsch Med. Wochenschr. 2005; 130(33):1891-6). Fullfillment of the ACR-criteria for RA was assessed after one year of follow-up.

The third validation cohort consisted of patients included in the placebo-arm of the Dutch PROMPT-trial, a double blind placebo-controlled randomized trial in which patients with recent-onset UA were treated with either methotrexate or placebo. (van Dongen et al., Arthritis Rheum. 2007; 56(5): 1424-32). Of the 36 independent UA-patients, two were lost-to-follow-up. This cohort was used previously for validation of the original prediction rule. (van der Helm-van Mil et al., Arthritis Rheum. 2007; 56(2):433-40).

All studies were approved by the local ethical committees and all patients gave written informed consent to participation in the studies.

Original Prediction Model

The original prediction model in the Leiden EAC cohort study is based on using the assigned risk values for the clinical parameter values shown in Table 1 at p. 19-20. In the original prediction rule, the presence or absence of anti-CCP2 antibodies is determined and a maximal score or risk value of 2 is assigned if anti-CCP2 antibodies are present. The presence or absence of anti-MCV antibodies were not determined in the original study. In addition, the morning stiffness severity (measured as Length of VAS) is used in additional to the other clinical parameter values shown. The maximal total prediction score for the clinical parameters values in the original prediction model is 14.

The predicted risk of developing RA as a function of the total risk values in the original prediction model is described in FIG. 5 above.

Re-Derived Prediction Model

The prediction rule was “Re-derived” in the Leiden EAC cohort study with the morning stiffness duration substituted for the morning stiffness severity in the original study. The maximal score for the duration of morning stiffness is now 1 (compared to 2 in the original prediction rule) as shown in Table 1 at p. 19-20. Consequently, the maximal total prediction score is now 13 instead of 14.

Enhanced Prediction Model

The prediction model in the re-derived parameters is “enhanced” in the same Leiden EAC cohort study as a further method for predicting whether the individual with UA will develop RA. The “enhanced” parameter includes determining the presence or absence of anti-MCV antibodies as a further clinical parameter, for example, a risk value of 2 can be assigned to anti-MCV antibody levels of >20 U/mL or alternatively, a risk value of 1 is assigned if either anti-MCV or anti-CCP3 antibodies are tested positive in the samples or a risk value of 2.5 is assigned if both anti-MCV and anti-CCP3 antibodies are tested positive in the samples. In addition, the risk values for other clinical parameters have been reassigned as shown in col. 4 of Table 1 at p. 19-20. Consequently, the maximal total prediction score is 8.5. In this model, the presence or absence of RF autoantibodies are not determined. In other models, the prediction score and hence risk of developing RA is calculated by omitting other clinical parameters such as involvement of tender and/or swollen joints with and without detecting levels of CRP.

Table 4 below shows the sensitivity and specificity values of anti-CCP3 and anti-MCV antibodies.

TABLE 4
Sensitivity and Specificity Values of
Anti-CCP3 and Anti-MCV Antibodies

| | Anti-CCP3.1 Ab. | Anti-MCB Ab. |
|---|---|---|
| Sensitivity | 60% | 62% |
| Specificity | 85% | 79% |
| Positive Predictive Value (PPV) | 66% | 59% |
| Negative Predictive Value (NPV) | 81% | 81% |

### Statistical Analysis

Data reported herein include the mean±SD and in case of skewed distribution as median and interquartile range. Differences in means between groups were analyzed with the Mann-Whitney test. Proportions were compared using the chi-square test. The re-derived prediction rule substitutes the duration of morning stiffness for the severity of morning stiffness was performed using logistic regression analysis. To get a simplified prediction rule, the regression coefficients of the predictive variables were rounded to the nearest number ending in 0.5 or 0 resulting in a weighted score. For all individual patients in the different cohorts the prediction score was calculated using the baseline patient characteristics.

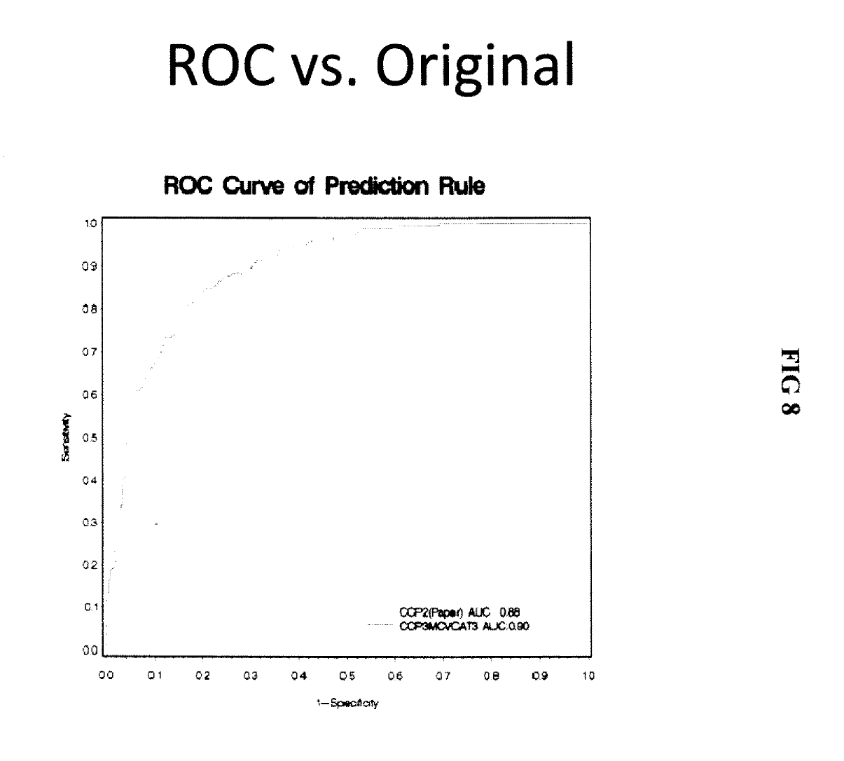

The prediction score and actual outcomes were compared. FIG. 7 shows the predicted risk of developing RA as a function of the total risk values where the duration of morning stiffness is substituted for the morning stiffness severity and the presence or absence of anti-MCV, anti-CCP3 and both are determined and assigned risk values. FIG. 7 shows the predicted risk curve superimposed on the predicted risk curve obtained in FIG. 5.

The positive and negative predictive values (PPV, NPV respectively, where PPV indicates the percent of patients studied who progressed to develop RA and NPV indicates the percent of patient who did not progress to develop RA) were determined for several cut-off values of the prediction score. For example, the NPV cut-off value used in this study is ≦6 and the PPV cut off value used is ≧8.

A receiver-operator characteristic (ROC) curve was constructed to evaluate the diagnostic performance and the area under the curve (AUC) provides a measure of the overall discriminative ability of the prediction rule. FIG. 8 shows the ROC of the prediction rule of the “Enhanced” compared to the “Original”. As shown in FIG. 8, the ROC using anti-CCP2 antibody (“Original”) is identical to that when anti-CCP3/anti-MCV/antibodies (including both) are used.

The Statistical Package for Social Sciences (SPSS), version 12.0 (Chicago, Ill.) was used. P-values <0.05 were considered significant.

### Results

### Validation Cohorts

Baseline characteristics of the early UA-patients are presented in Table 5. Consistent with the different inclusion criteria of the cohorts, the symptom duration differed accordingly with the lowest symptom duration in the Birmingham cohort (mean 41 days) and the highest symptom duration in the Dutch cohort (mean 327 days). The three cohorts differed in the patient characteristics that constitute the prediction rule; consequently the total prediction score is different for the three groups (Birmingham vs. Berlin cohort p=0.007, other comparisons NS). The percentage of patients that progressed to RA was 31% in the Birmingham cohort, 37% in the Berlin cohort and 44% in the Dutch cohort.

TABLE 5
Baseline characteristics of different cohorts of early UA-patients

| | Birmingham, UK | Berlin, Germany | Dutch PROMPT |
|---|---|---|---|
| | N = 99 | N = 155 | N = 34 |
| Age mean ± SD | 48.2 ± 16.4 | 50.8 ± 14.8 | 51.6 ± 12.4 |
| Female gender, No (%) | 60 (61%) | 113 (73%) | 28 (78%) |
| Symptom duration, days, | 41 ± 25 | 131 ± 96 | 327 ± 198 |
| mean ± SD | | | |
| Number tender joints, | 5.4 ± 6.8 | 7.6 ± 7.6 | 6.8 ± 6.1 |
| mean ± SD | | | |
| Number swollen joints, | 3.3 ± 3.5 | 3.5 ± 5.2 | 3.1 ± 6.7 |
| mean ± SD | | | |

Distribution of involved joints, No (%)

| | | | |
|---|---|---|---|
| Symmetric | 40 (40%) | 89 (57%) | 12 (33%) |
| Small joints involved | 49 (49%) | 110 (71%) | 28 (78%) |
| Upper extremities | 40 (40%) | 110 (71%) | 28 (78%) |
| Upper + lower extremities | 16 (16%) | 97 (63%) | 13 (36%) |
| Duration of morning | 66.0 ± 76.4 | 24.2 ± 45.5 | 44.4 ± 57.0 |
| stiffness (min), mean ± SD | | | |
| CRP (mg/L), median (IQR) | 23.0 (7.0, 54.0) | 6.8 (2.1, 18.3) | 3.0 (3.0, 6.0) |
| RF positive, No (%) | 17 (17%) | 72 (46%) | 11 (31%) |
| Anti-CCP positive, No (%) | 12 (12%) | 35 (23%) | 8 (22%) |
| Total prediction score, | 4.7 ± 2.3 | 5.6 ± 2.3 | 5.7 ± 2.2 |
| mean ± SD | | | |
| Progression to RA, No (%) | 31 (31%) | 58 (37%) | 15 (44%) |

Table 6 shows the predictive values and discriminative ability based on the baseline characteristics of the three different cohort studies of early UA patients using the cut-off values of ≦6 and ≧8 for NPV and PPV respectively. Based the clinical parameters shown in Table 5, about 25% of patients were in the intermediate group (score between 6 and 8) for whom no accurate prediction could be made.

TABLE 6
Predictive values and discriminative ability

| | Re-derived prediction | Birmingham | Berlin | Dutch | Three validation |
|---|---|---|---|---|---|
| | rule Leiden EAC | UK | Germany | PROMPT | cohorts combined |
| | N = 570 | N = 99 | N = 155 | N = 34 | N = 288 |
| NPV of score ≦6 | 89% | 82% | 83% | 86% | 83% |
| PPV of score ≧8 | 82% | 100% | 93% | 100% | 97% |
| Proportion patients | 24% | 27% | 22% | 24% | 24% |
| with score 6-8 | | | | | |
| AUC (SE) | 0.88 (0.015) | 0.83 (0.041) | 0.82 (0.037) | 0.95 (0.031) | 0.84 (0.024) |

In the Birmingham cohort (See, column 3, Table 6 above), 54 out of 65 patients (NPV=82%) with a score ≦6 did not develop RA, all seven patients with a score ≧8 progressed to RA (PPV=100%) and 27 patients (27%) had a score between 6 and 8. The AUC was 0.83 (SE 0.041).

In the Berlin cohort (See, column 4, Table 6 above), 78 of the 91 patients (NPV=83%) with a score ≦6 did not progress to RA, 25 of the 27 patients (PPV=93%) with a score ≧8 were diagnosed with RA and 34 (22%) had a score in between 6 and 8. The AUC in this cohort was 0.82 (SE 0.037).

In the Dutch replication cohort (See, column 5, Table 6 above), 18 out of 21 patients with a score ≦6 did not progress to RA (NPV=86%), all 5 patients with a score ≧8 developed RA (PPV=100%) and 8 patients (24%) had an intermediate score between 6 and 8. The AUC in this cohort was 0.95 (SE 0.031).

Combining the three cohorts resulted in a combined PPV of 97%, a combined NPV of 83% and an combined AUC of 0.84 (SE 0.024) (See, column 6, Table 6 above). The diagnostic performances visualized as the receiver operator characteristic curve of the derivation cohort as well as of the three validation cohorts are presented in Table 6.

The different baseline characteristics between patients in the three cohorts may be due to different inclusion criteria, in particular the maximum permissible symptom duration at entry. However, these patient cohorts represent a broad cross-section of early UA-patients and the prediction model accurately estimated the disease outcome in all three cohorts.

The present study assessed the predictive accuracy of a original and derived prediction model by estimating the chance of progression to RA in three independent cohorts of early UA-patients. In all replication cohorts, the positive and negative predictive values as well as the area under the receiver operator curve were only marginally lower than those in the derivation cohort. The observation of accurate predictions in several independent cohorts of early UA-patients, originating from different countries, demonstrates the discriminative ability and validity of the prediction model and provides the foundation for the use of this rule in clinical practice.

Results from Re-derived and Enhanced Prediction Models from the Leiden EAC Study

As discussed above, the prediction rule was re-derived in the Leiden EAC with the morning stiffness duration substituted for the morning stiffness severity. The NPV and PPV of the re-derived prediction score were assessed with the cut-off values ≦6 and ≧8.

For the re-derived and enhanced models, the severity of morning stiffness was not recorded in either the Birmingham or Berlin cohorts, but the duration of morning stiffness was. In addition, the enhanced model included the determination of anti-MCV antibodies in addition to anti-CCP3 antibodies. The results are shown in Table 7 below.

TABLE 7
Comparison of the Original, Re-derived and Enhance Predictive Rule

| | Original | Re-derived | Enhanced |
|---|---|---|---|
| Parameter | (N = 570) | (N = 570) | (N = 499) |
| NPV | 91% | 89% | 88% |
| PPV | 84% | 82% | 82% |
| % unclassified | 25% | 24% | 15% (middle 6-7) |
| AUC-ROC + std. | 0.89 ± 0.014 | 0.88 ± 0.015 | 0.90 ± 0.014 |
| error | | | |

Table 7 shows that 89% of patients with a score ≦6 did not develop RA (compared to 91% in the original prediction rule), 82% of patients with a score ≧8 progressed to RA (compared to 84% in the original prediction rule) and 24% remained unclassified (compared to 25% in the original prediction rule). The AUC of the re-derived prediction rule was 0.88 (SE 0.015), which is slightly lower than in the original prediction rule (AUC 0.89, SE 0.014). Thus, the accuracy of the original prediction rule and re-derived prediction rule were only slightly different (AUC 0.88 and 0.89 respectively).

When the “enhanced” prediction model is used for predicting the risk of the patients having UA developing RA for the same cohort, 88% of patients with a score ≦6 did not develop RA (compared to 91% in the original prediction rule and 89% in the re-derived), 82% of patients with a score ≧8 progressed to RA (compared to 84% in the original prediction rule and 82% in the re-derived) and only 15% remained unclassified (compared to 25% in the original prediction rule and 24% in the re-derived). The AUC of the re-derived prediction rule was 0.90 (SE 0.014), which is slightly lower than in the original prediction rule (AUC 0.89, SE 0.014).

With the original prediction model no adequate prediction can be made in a quarter of the patients (the patients with a score between 6 and 8). The proportion of these patients was comparable in the derivation cohort model and all three validation cohorts. However, with the “enhanced” prediction model, only 15% of the patients remained unclassified compared to 25% in the original prediction rule and 24% in the re-derived.

Data on radiological joint destruction or on genetic risk factors for RA (HLA-DRB1 shared epitope alleles, PTPN22, C5-TRAF) were studied in the derivation cohort and were found not to be independent predictors for RA-development in logistic regression analysis. Therefore, these variables were of no additive value for the patients with a score between 6 and 8. Further, misclassification may have occurred when patients who presented with UA were treated with a drug that may have slowed the rate of progression to RA. Patients whose natural history would have been progression to RA may, with treatment, not have accrued sufficient features to allow their classification as RA. Disease Modifying Anti-Rheumatic Drugs (DMARDs) were started in 22% (Birmingham cohort) and 25% (Berlin cohort) of the UA-patients who did not progress to RA. In the Dutch replication cohort no DMARDs were used. Such patient misclassification would mean that the predictive values of the current model and the AUC of this model are underestimates.

In addition to the “enhanced” predictive model, alternative models were developed by omitting certain clinical markers and/or clinical parameter values. These clinical markers and/or parameter values omitted in the “alternative” predicting of risk of RA include, for example, omitting the test for the presence or absence of RF, CRP and/or involvement of tender and swollen joints. The results from these alternative models based on the “enhanced” predictive model is shown in Table 8 below.

TABLE 8
Alternative Models for “Enhanced” Predictive Rule

| | | | | Without Tender and |
|---|---|---|---|---|
| | | | Without Tender and | Swollen Joints and |
| | Enhanced | Without RF | Swollen Joints | CRP and RF |
| Parameter | (N = 499) | (N = 499) | (N = 499) | (N = 499) |
| NPV | 88% | 89% | 86% | 86.6% |
| PPV | 82% | 79% | 84% | 77% |
| % unclassified | 15% | 13% | 16% | 17% |
| (middle 6-7) | | | | |
| AUC-ROC + std. | 0.90 ± 0.014 | 0.90 ± 0.014 | 0.885 ± 0.015 | 0.87 ± 0.016 |
| error | | | | |

The result of the “enhanced” model with the exclusion of 1) number of tender joints and 2) number of swollen joints (see the forth column in Table 8) is the same as the result of the “enhanced” model with the exclusion of 1) RF autoantibodies, 2) number of tender joints, and 3) number of swollen joints. Similarly the result of the “enhanced” model with the exclusion of 1) number of tender joints, 2) number of swollen joints, 3) level of CRP, HS-CRP or ESR and 4) RF autoantibodies is the same as the result of the “enhanced” model with the exclusion of 1) number of tender joints, 2) number of swollen joints, 3) localization categorical for tender/swollen joints, 4) level of CRP, HS-CRP or ESR and 5) RF autoantibodies

The current prediction model appears to be the first validated for patients with early undifferentiated arthritis and it should facilitate the development of personalized medicine in this clinical context. There is widespread interest in the development of predictive tools in other clinical situations. The descriptive ability, as measured by the AUC, using the prediction model described herein is better than that of currently available predictive tools, many of which require additional or invasive measurements. In contrast, the information needed to use the present prediction model for early undifferentiated arthritis is easily and regularly collected at the first visit to the clinic. The prediction model described herein accurately estimates the risk of developing RA in more than 75% of individual patients with recent-onset UA.

All publications, patents and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art. It will be appreciated, however, that no matter how detailed the foregoing appears in text, the invention can be practiced in many ways.

Unless defined otherwise, all technical and scientific terms used herein have the same meaning as commonly understood by one of ordinary skill in the art to which this invention belongs. It should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Each of the following references is incorporated by reference in their entireties for all purposes.
1. Lossos I S, Czerwinski D K, Alizadeh A A, et al. Prediction of survival in diffuse large-B-cell lymphoma based on the expression of six genes. N Engl J. Med. 2004; 350(18):1828-37.
2. Hasenclever D, Diehl V. A prognostic score for advanced Hodgkin's disease. International Prognostic Factors Project on Advanced Hodgkin's Disease. N Engl J. Med. 1998; 339(21): 1506-14.
3. Lard L R, Visser H, Speyer I, et al. Early versus delayed treatment in patients with recent-onset rheumatoid arthritis: comparison of two cohorts who received different treatment strategies. Am J. Med. 2001; 111(6):446-51.
4. Goekoop-Ruiterman Y P, De Vries-Bouwstra J K, Allaart C F et al. Clinical and radiographic outcomes of four different treatment strategies in patients with early rheumatoid arthritis (the BeSt study): A randomized, controlled trial. Arthritis Rheum 2005; (11):3381-90.
5. Nell V, Machold K P, Eberl G, Stramm T A, Uffman M, Smolen J S. Benefit of very early referral and very early therapy with disease-modifying antirheumatic drugs in patients with early rheumatoid arthritis. Rheumatology (oxford) 2004; 43:906-14.
6. van Aken J, Van Dongen H, le Cessie S, Allaart C F, Breedveld F C, Huizing a T W. Long-term outcome of rheumatoid arthritis that presented with undifferentiated arthritis compared to rheumatoid arthritis at presentation—an observational cohort study. Ann Rheum Dis. 2006; 65(1):20-5.
7. Tunn E J, Bacon P A. Differentiating persistent from self-limiting symmetrical synovitis in an early arthritis clinic. Br J. Rheumatol. 1993; 32(2):97-103.
8. Harrison B J, Symmons D P, Brennan P, Barrett E M, Silman A J. Natural remission in inflammatory polyarthritis: issues of definition and prediction. Br J. Rheumatol. 1996; 35(11):1096-100.
9. van Dongen H, van Aken J, Lard L R, et al. Treatment of Patients with Undifferentiated Arthritis with Methotrexate: a Double-Blind, Placebo-Controlled, Randomized Clinical Trial to Prevent Evolvement into RA. ACR 2005, abstract (479)L4.
10. Machold K P, Stamm T A, Eberl G J, et al. Very recent onset arthritis-clinical, laboratory, and radiological findings during the first year of disease. J. Rheumatol. 2002; 29(11):2278-87.
11. van Aken J, van Bilsen J H, Allaart C F, Huizing a T W, Breedveld F C. The Leiden Early Arthritis Clinic. Clin Exp Rheumatol. 2003; 21(5 Suppl 31):S100-5.
12. Hazes J M, Hayton R, Silman A J. A re-evaluation of the symptom morning stiffness. J Rheumatol 1993; 20:1138-42.
13. Vliet Vlieland Th P, Zwinderman A H, Breedveld F C, Hazes J M. Measurement of morning stiffness in rheumatoid arthritis clinical trials. J Clin Epidemiol 1997; 50:757-63.
14. Van Riel P L, van Gestel A M, Scott D G. In EULAR handbook of clinical assessments in rheumatoid arthritis. Alphen aan den Rijn, The Netherlands: Van Zuiden Communications; 2000, 10-11.
15. Van der Heijde D M. Plain X-rays in rheumatoid arthritis: overview of scoring methods, their reliability and applicability. Baillieres Clin Rheumatol. 1996; 10:435-53.
16. Harrell F E, Lee K L, Mark D B. Multivariate prognostic models, evaluating assumptions and accuracy, and measuring and reducing errors. Stat Med. 1996; 15:361-87.
17. Visser H, le Cessie S, Vos K, Breedveld F C, Hazes J M. How to diagnose rheumatoid arthritis early: a prediction model for persistent (erosive) arthritis. Arthritis Rheum. 2002; 46(2):357-65.
18. Harrison B J, Symmons D P, Barrett E M, Silman A J. The performance of the 1987 ARA classification criteria for rheumatoid arthritis in a population based cohort of patients with early inflammatory polyarthritis. American Rheumatism Association. J. Rheumatol. 1998; 25(12):2324-30.
19. Harrison B, Symmons D. Early inflammatory polyarthritis: results from the Norfolk Arthritis Register with a review of the literature. II. Outcome at three years. Rheumatology (Oxford). 2000; 39(9):939-49.
20. Symmons D P, Hazes J M, Silman A J. Cases of early inflammatory polyarthritis should not be classified as having rheumatoid arthritis J. Rheumatol. 2003; 30(5):902-4.
21. Green M, Marzo-Ortega H, McGonagle D, et al. Persistence of mild, early inflammatory arthritis: The importance of disease duration, rheumatoid factor, and the shared epitope. Arthritis Rheum. 1999; 42:2184-88.
22. Linn-Rasker S P, Allaart C F, Kloppenburg M, Breedveld F C, Huizing a TWJ. Sustained remission in a cohort of patients with RA: association with absence of IgM-rheumatoid factor and absence of anti-CCP antibodies. Int J Advances Rheumatology. 2004; 2 (4):4-6.
23. van der Helm-van Mil A H, Dieude P, Schonkeren J J, Cornelis F, Huizing a T W. No association between tumour necrosis factor receptor type 2 gene polymorphism and rheumatoid arthritis severity: a comparison of the extremes of phenotypes. Rheumatology (Oxford). 2004; 43(10): 1232-4.
24. Aletaha D, Breedveld F C, Smolen J S. The need for new classification criteria for rheumatoid arthritis. Arthritis Rheum. 2005:52:3333-36.
25. Visser H. Early diagnosis of rheumatoid arthritis. Best Practice & Research Clinical Rheumatology. 2005:19(1):55-72.
26. Morel J, Combe B. How to predict prognosis in early rheumatoid arthritis. Best Practice & Research Clinical Rheumatology. 2005:19(1):137-46.

1. A method of predicting whether an individual with undifferentiated arthritis will develop rheumatoid arthritis comprising
determining for the individual the presence or absence of anti-MCV antibody, wherein the presence of anti-MCV antibody in the individual is indicative of the risk of the individual for developing rheumatoid arthritis.

2. The method of claim 1, further comprising determining the duration of morning stiffness of the individual, wherein the duration of morning stiffness correlates with the risk of the individual for developing rheumatoid arthritis.

3. The method of claim 1, further comprising
determining at least one clinical parameter of the individual selected from the group consisting of 1) age, 2) gender, 3) distribution of involved joints, 4) duration of morning stiffness, 5) number of tender joints, and 6) number of swollen joints,
assigning a risk value for the clinical parameter based on a predefined risk value index for the clinical parameter, and
predicting the risk of the individual of developing rheumatoid arthritis based on the presence or absence of anti-MCV antibody in combination with the risk value of the clinical parameter.

4. The method of claim 1, further comprising
determining the presence or absence of anti-CCP antibody or Rheumatoid factor autoantibody, wherein the presence of anti-CCP antibody or Rheumatoid factor autoantibody correlates with the risk of the individual for developing rheumatoid arthritis.

5. The method of claim 1, further comprising
determining the serum level of a clinical marker selected from the group consisting of C-reactive protein (CRP), high sensitivity C-reactive protein (HS CRP) and erythrocyte sedimentation rate (ESR),
assigning a risk value for the level of the clinical marker based on a predefined risk value index for the clinical marker, and
predicting the risk of the individual of developing rheumatoid arthritis based on the presence or absence of anti-MCV antibody in combination with the risk value of the clinical marker.

6. The method of claim 1, further comprising
determining a set of clinical parameters,
assigning a risk value for each clinical parameter based on a predefined risk value index for each clinical parameter,
assigning a predefined risk value for the presence or absence of anti-MCV antibody in the individual, and
predicting the risk of the individual of developing rheumatoid arthritis based on the collection of the risk value for each clinical parameter in combination with the presence or absence of anti-MCV antibody,
wherein the set of clinical parameters comprises 1) age, 2) gender, 3) distribution of involved joints, and 4) duration of morning stiffness.

7. The method of claim 6, wherein the set of clinical parameters comprises 1) age, 2) gender, 3) distribution of involved joints, 4) duration of morning stiffness, 5) number of tender joints, and 6) number of swollen joints.

8. The method of claim 6, further comprising
determining the presence or absence of a clinical marker selected from the group consisting of anti-CCP antibody and Rheumatoid factor autoantibody,
assigning a predefined risk value to the presence or absence of the clinical marker, and
predicting the risk of the individual of developing rheumatoid arthritis based on the collection of the risk value for each clinical parameter, clinical marker, and the presence or absence of anti-MCV antibody.

9. The method of claim 6, further comprising
determining the serum level of a clinical marker selected from the group consisting of C-reactive protein (CRP), high sensitivity C-reactive protein (HS CRP) and erythrocyte sedimentation rate (ESR),
assigning a risk value for the level of the clinical marker based on a predefined risk value index for the clinical marker, and
predicting the risk of the individual of developing rheumatoid arthritis based on the collection of the risk value for each clinical parameter, clinical marker, and the presence or absence of anti-MCV antibody.

10. The method of claim 6, further comprising
determining the presence or absence of a first clinical marker selected from the group consisting of anti-CCP antibody and Rheumatoid factor autoantibody,
assigning a predefined risk value to the presence or absence of the first clinical marker,
determining the serum level of a second clinical marker selected from the group consisting of C-reactive protein (CRP), high sensitivity C-reactive protein (HS CRP) and erythrocyte sedimentation rate (ESR),
assigning a risk value for the level of the second clinical marker based on a predefined risk value index for the clinical marker, and
predicting the risk of the individual of developing rheumatoid arthritis based on the collection of the risk value for each clinical parameter, clinical marker, and the presence or absence of anti-MCV antibody.

11. The method of claim 10, wherein the predefined risk value is selected from the group consisting of
1) 0.03 for each year of age,
2) 0 for male gender and 0.5 for female,
3) 0.5 in case of involvement of small joints hands and feet, symmetric or upper extremities involvement, and 1 in case of upper and lower extremities involvement,
4) 0.5 in case of 30-59 minute morning stiffness and 1 in case of 60 minutes or more morning stiffness,
5) 0.5 for 4-10 tender joints and 1 for 11 or more tender joints,
6) 0.5 for 4-10 swollen joints and 1 for 11 or more tender joints,
7) 0.5 for 5-50 mg/L CRP and 1 for 51 mg/L or higher CRP,
8) 0 for the absence of Rheumatoid factor autoantibody and 1 for the presence of Rheumatoid factor autoantibody, and
9) 0 for the absence of anti-MCV antibody or anti-CCP antibody while 1 for the presence of anti-MCV antibody or anti-CCP antibody, and 2.5 for the presence of anti-MCV antibody and anti-CCP antibody.

12. The method of claim 1, wherein the individual is an individual with recent onset undifferentiated arthritis or with a presumed but unconfirmed diagnosis of rheumatoid arthritis.

13. A computer comprising a processor and a memory, the processor being arranged to read from said memory and write into said memory, the memory comprising data and instructions arranged to provide said processor with the capacity to perform the method of claim 6.

14. A system for determining a predicted risk of an individual with undifferentiated arthritis to develop rheumatoid arthritis comprising
a) means for receiving a characteristic of a clinical parameter selected from the group consisting of 1) age, 2) gender, 3) distribution of involved joints, 4) duration of morning stiffness, 5) number of tender joints, and 6) number of swollen joints,
b) means for receiving a characteristic of a first clinical marker comprising anti-MCV antibody and optionally a second clinical marker selected from the group consisting of anti-CCP antibody, Rheumatoid factor autoantibody, C-reactive protein (CRP), high sensitivity C-reactive protein (HS CRP) and erythrocyte sedimentation rate (ESR),
c) means for assigning a risk value to each characteristic of the clinical parameter and the clinical marker; and
d) means for determining a predicted risk of the individual developing rheumatoid arthritis based at least partly on the assigned risk values.

15. A system for determining a predicted risk of an individual with undifferentiated arthritis developing rheumatoid arthritis, the system comprising:
a) a blood sample analyzer configured to analyze a blood sample of the individual and determine the presence or absence of a first clinical marker of anti-MCV antibody, and optionally a second clinical marker selected from the group consisting of anti-CCP antibody, Rheumatoid factor autoantibody, C-reactive protein (CRP), high sensitivity C-reactive protein (HS CRP) and erythrocyte sedimentation rate (ESR); and
b) a computing device configured to assign a risk value to each of the clinical marker determined by the blood sample analyzer based on predefined risk values associated with each clinical marker stored in a memory, and to determine a predicted risk of the individual developing rheumatoid arthritis based at least partly on the collection of the risk value assigned to each of the clinical marker.

16. A combination of tests useful for predicting whether an individual with undifferentiated arthritis will develop rheumatoid arthritis comprising
a first test for the presence or absence of anti-MCV antibodies and
a second test selected from the group consisting of tests for the serum level of C-reactive protein, HS-CRP or ESR, tests for the presence or absence of Rheumatoid factor autoantibody, and tests for the presence or absence of anti-CCP antibodies.

17. The combination of claim 16, comprising
a first test for the presence or absence of anti-MCV antibodies,
a second test for the serum level of C-reactive protein, HS-CRP or ESR,
a third test for the presence or absence of Rheumatoid factor autoantibody, and
a fourth test for the presence or absence of anti-CCP antibodies.

18. The combination of claim 16, wherein the first test for the presence or absence of anti-MCV antibodies includes using a peptide derived from native vimentin and comprising at least one additional arginine residue compared to the native sequence.

19. The combination of claim 16, wherein the first test for the presence or absence of anti-MCV antibodies includes using a peptide derived from native vimentin and comprising at least one additional arginine residue in at least one of positions 16, 17, 19, 41, 58, 59, 60, 68, 76, 140, 142, 147, 363, 406 or 452.

20. A combination of tests useful for predicting whether an individual with undifferentiated arthritis will develop rheumatoid arthritis comprising at least three tests selected from the group consisting of tests for the presence or absence of anti-MCV antibodies, tests for the serum level of C-reactive protein, HS-CRP or ESR, tests for the presence or absence of Rheumatoid factor autoantibody, and tests for the presence or absence of anti-CCP antibodies.

21. The combination of claim 20, wherein tests for the presence or absence of anti-MCV antibodies include using a peptide derived from native vimentin and comprising at least one additional arginine residue compared to the native sequence.

22. The combination of claim 20, wherein tests for the presence or absence of anti-MCV antibodies include using a peptide derived from native vimentin and comprising at least one additional arginine residue in at least one of positions 16, 17, 19, 41, 58, 59, 60, 68, 76, 140, 142, 147, 363, 406 or 452.

23. A method of providing useful information for predicting whether an individual with undifferentiated arthritis will develop rheumatoid arthritis comprising
determining a set of clinical markers for the individual and
providing the set of clinical markers to an entity that combines the set of clinical markers with a set of clinical parameters to provide the prediction,
wherein the set of clinical markers include the presence or absence of anti-MCV antibodies and at least one clinical marker value selected from the group consisting of the serum level of C-reactive protein, HS-CRP or ESR, the presence or absence of Rheumatoid factor autoantibody, and the presence or absence of anti-CCP antibodies.

24. The method of claim 23, wherein the set of clinical markers include the presence or absence of anti-MCV antibodies, the serum level of C-reactive protein, HS-CRP or ESR, the presence or absence of Rheumatoid factor autoantibody, and the presence or absence of anti-CCP antibodies.

25. The method of claim 23, wherein the set of clinical parameters include the duration of morning stiffness of the individual.

26. The method of claim 23, wherein the set of clinical parameters include at least two clinical parameters selected from the group consisting of the duration of morning stiffness of the individual, the age of the individual, the gender of the individual, the localization of the joint complaints of the individual, the number of tender joints of the individual, and the number of swollen joints of the individual.

27. The method of claim 23, wherein the presence or absence of anti-MCV antibodies is detected via using a peptide derived from native vimentin and comprising at least one additional arginine residue compared to the native sequence.

28. The method of claim 23, wherein the presence or absence of anti-MCV antibodies is detected via using a peptide derived from native vimentin and comprising at least one additional arginine residue in at least one of positions 16, 17, 19, 41, 58, 59, 60, 68, 76, 140, 142, 147, 363, 406 or 452.

29. The method of claim 23, wherein the entity is a clinician or a service provider.

30. A collection of results useful for predicting whether an individual with undifferentiated arthritis will develop rheumatoid arthritis comprising values for a first set of clinical markers for the individual, wherein the first set of clinical markers include the presence or absence of anti-MCV antibodies and at least one clinical marker value selected from the group consisting of the serum level of C-reactive protein, HS-CRP or ESR, the presence or absence of Rheumatoid factor autoantibody, and the presence or absence of anti-CCP antibodies.

31. The collection of results of claim 30, wherein the first set of clinical markers include the presence or absence of anti-MCV antibodies, the serum level of C-reactive protein, HS-CRP or ESR, the presence or absence of Rheumatoid factor autoantibody, and the presence or absence of anti-CCP antibodies.

32. The collection of results of claim 30, wherein the presence or absence of anti-MCV antibodies is detected via using a peptide derived from native vimentin and comprising at least one additional arginine residue compared to the native sequence.

33. The collection of results of claim 30, wherein the presence or absence of anti-MCV antibodies is detected via using a peptide derived from native vimentin and comprising at least one additional arginine residue in at least one of positions 16, 17, 19, 41, 58, 59, 60, 68, 76, 140, 142, 147, 363, 406 or 452.

34. The collection of results of claim 30, further comprising an instruction for using the values for the first set of clinical markers in combination with a set of clinical parameters for the individual, wherein the set of clinical parameters include the duration of morning stiffness of the individual.

35. The collection of results of claim 30, further comprising an instruction for using the values for the first set of clinical markers in combination with a set of clinical parameters for the individual, wherein the set of clinical parameters include at least two clinical parameters selected from the group consisting of the duration of morning stiffness of the individual, the age of the individual, the gender of the individual, the localization of the joint complaints of the individual, the number of tender joints of the individual, and the number of swollen joints of the individual.
